(19) United States
(12) Patent Application Publication
(10) Pub. No.: US 2024/0100011 A1
(43) Pub. Date: Mar. 28, 2024

(54) PEDIATRIC FORMULATIONS OF FERRIC CITRATE

(71) Applicant: Keryx Biopharmaceuticals, Inc., Cambridge, MA (US)

(72) Inventors: Dragutin KNEZIC, Arlington, MA (US); Aniruddh PATEL, North Grafton, MA (US); Farzaneh SEYEDI, Mansfield, MA (US); Anandhavalavan ARULMOZHI, Quincy, MA (US); Milind DIXIT, Georgetown (CA)

(73) Assignee: Keryx Biopharmaceuticals, Inc., Cambridge, MA (US)

(21) Appl. No.: 18/517,274

(22) Filed: Nov. 22, 2023

Related U.S. Application Data
(63) Continuation of application No. PCT/US2022/031239, filed on May 27, 2022.
(60) Provisional application No. 63/193,938, filed on May 27, 2021.

(51) Int. Cl. A61K 31/295; A61K 9/28; A61K 47/14; A61K 47/16

(52) U.S. Cl. CPC A61K 31/295; A61K 9/2866; A61K 47/14; A61K 47/16

(57) ABSTRACT

Described herein are ferric citrate-containing pharmaceutical compositions (e.g., solid oral dosage forms such as tablets). The pharmaceutical compositions described here can be administered to a subject in need thereof. In particular, the pharmaceutical compositions described here can be administered to a subject who is ≤ about 18 years of age (e.g., about 6-18 years of age or about 12 to 17 years of age).

### CROSS-REFERENCE TO RELATED APPLICATIONS

The present application claims benefit of U.S. Provisional Application No. 63/193,938, filed May 27, 2021, which is hereby incorporated by reference in its entirety.

### BACKGROUND OF THE INVENTION

Ferric citrate can be therapeutically beneficial for various diseases and disorders, including the control of phosphate metabolism and prevention of metabolic acidosis in patients as well as treatment or prevention of iron deficiency and/or anemia. For example, ferric citrate compounds can be administered to patients suffering from kidney disease or renal failure, including in order to treat or prevent conditions such as hyperphosphatemia, iron deficiency, and/or anemia. However, considerable challenges (e.g., palatability issue due to metallic taste, and probable staining of the teeth) are presented in the development of formulations suitable for pediatric populations, for example, a subject who is ≤ about 18 years of age (e.g., about 6-18 years of age). Accordingly, there remains an unmet need for formulations of ferric citrate suitable for pediatric patients.

### SUMMARY

In part, the present invention provides ferric citrate-containing pharmaceutical compositions (e.g., solid oral dosage forms such as tablets) which can be administered to a subject in need thereof. In particular, the pharmaceutical compositions described here can be administered to a subject who is ≤ about 18 years of age (e.g., about 6-18 years of age).

For example, described herein are ferric citrate-containing pharmaceutical compositions (e.g., solid oral dosage forms) comprising intragranular components (e.g., ferric citrate, binders, disintegrants, fillers or lubricants) and extragranular components (e.g., glidants or lubricants). Also provided are methods for their use in treating and/or preventing a disease or medical conditions, and methods for their preparation.

In particular, pharmaceutical compositions described herein can be administered to a subject in need thereof (e.g., for the prophylaxis or treatment of hyperphosphatemia, or for the treatment of iron deficiency anemia). For example, the described pharmaceutical compositions may be administered to subjects who is ≤ about 18 years of age.

Exemplary ferric citrate-containing pharmaceutical compositions and exemplary methods are described herein.

In one aspect, the present invention provides herein a pharmaceutical composition formulated as a solid oral dosage form, comprising:
an intragranular component comprising
   ferric citrate present in an amount that is about 60-80 weight %;
   one or more binders present in a total amount that is about 1-10 weight %;
   one or more disintegrants present in a total amount that is about 1-5 weight %;
   one or more fillers present in a total amount that is about 10-30 weight %; and
   one or more lubricants present in a total amount that is about 0.1-2 weight %; and
an extragranular component comprising
   one or more glidants present in a total amount that is about 0.1-2 weight %; and
   one or more lubricants present in a total amount that is about 0.1-2 weight %;
wherein the weight % is determined based on the total weight of the tablet.

In embodiments, one or more binders of the intragranular component are present in a total amount that is about 3-10, 3-9, 3-8, 3-7, 3-6, 3-5, 4-10, 4-9, 4-8, 4-7, or 4-6 weight %.

In embodiments, one or more binders of the intragranular component are selected from the group consisting of hydroxypropyl cellulose (HPC), hydroxypropylmethyl cellulose (HPMC), sodium alginate, alginic acid, guar gum, acacia gum, xanthan gum, carbolpol, cellulose gum (carboxy methyl cellulose), ethyl cellulose, maltodextrin, PVP/VA, povidone, microcrystalline cellulose, starch (partially or fully pregelatinized starch), methyl cellulose, and copovidone.

In embodiments, an intragranular component comprises a binder that is copovidone.

In embodiments, an intragranular component comprises a binder that is hydroxypropylmethyl cellulose (HPMC).

In embodiments, one or more disintegrants of the intragranular component are present in a total amount that is about 1-2, 2-3, 3-4, or 4-5 weight %.

In embodiments, one or more disintegrants of the intragranular component are selected from the group consisting of croscarmellose sodium, crospovidone, sodium starch glycolate, starch, and microcrystalline cellulose.

In embodiments, one or more fillers of the intragranular component are present in a total amount that is about 10-25, 10-20, 15-25, 15-30, 20-30, or 20-25 weight %.

In embodiments, one or more fillers of the intragranular component are selected from microcrystalline cellulose, starches, partially pregelatinized starches, sorbitol powder, mannitol powder, lactose, hydroxypropyl methyl cellulose, hydroxypropyl cellulose, maltodextrins, dried glucose syrup, and dextrose mono & anhydrous.

In embodiments, one or more lubricants of the intragranular component are present in a total amount of about 0.1-1 weight %; and/or one or more lubricants of the extragranular component are present in a total amount of about 0.1-1 weight %.

In embodiments, one or more lubricants of the intragranular and/or extragranular components are selected from the group consisting of magnesium stearate, calcium stearate, sodium stearyl fumarate, polyethylene glycol, sodium lauryl sulfate, talc, mineral oil, leucine, and poloxamer.

In embodiments, an intragranular and/or an extragranular component comprise a lubricant that is magnesium stearate.

In embodiments, an intragranular and/or an extragranular component comprise a lubricant that is calcium stearate.

In embodiments, one or more glidants of the extragranular component are present in a total amount of about 0.1-1 weight %.

In embodiments, one or more glidants of the extragranular component are selected from the group consisting of hydrophilic fumed silica, colloidal silicon dioxide, starch, talc, and magnesium stearate.

In embodiments, an extragranular component comprises a glidant that is hydrophilic fumed silica.

In embodiments, an extragranular component comprises a glidant that is colloidal silicon dioxide.

In another aspect, the present invention provides herein a pharmaceutical composition comprising:
an intragranular component comprising
   ferric citrate present in an amount that is about 60-80 weight %;
   two or more excipients selected from the group consisting of copovidone, microcrystalline cellulose, and crospovidone, wherein said excipients are present in a total amount that is about 20-35 weight %; and
   magnesium stearate or calcium stearate present in an amount that is about 0.1-2 weight %; and
an extragranular component comprising
   hydrophilic fumed silica or colloidal silicon dioxide present in an amount that is about 0.1-2 weight %; and
   magnesium stearate or calcium stearate present in an amount that is about 0.1-2 weight %;
wherein the weight % is determined based on the sum weights of the total weight of the tablet.

In embodiments, an intragranular component comprises copovidone, microcrystalline cellulose, and crospovidone.

In embodiments, ferric citrate is present in an amount that is about 60-75, 65-80, 65-75, 70-80, or 70-75 weight %.

In embodiments, ferric citrate is present in an amount that is about 65-75 or 70-75 weight %.

In embodiments, a pharmaceutical composition comprises about 100-1000 mg ferric citrate.

In embodiments, a pharmaceutical composition comprises about 100-900, 100-800, 100-700, 100-600, 100-500, 100-400, 100-300, 100-200, 200-900, 200-800, 200-700, 200-600, 200-500, 200-400, 200-300, 300-900, 400-800, 400-700, 400-600, 400-500, 500-900, 500-800, 500-700, or 500-600 mg ferric citrate.

In embodiments, a pharmaceutical composition comprises about 100-500, 200-500, or 300-500 mg ferric citrate or about 50, 100, 150, 200, 250, 300, 350, 400, 450, or 500 mg ferric citrate.

In embodiments, a pharmaceutical composition comprises about 250 mg ferric citrate.

In embodiments, a pharmaceutical composition is formulated as a tablet.

In embodiments, a tablet further comprises a coating.

In embodiments, a coating comprises hydroxypropyl methylcellulose (HPMC) as the binder.

In embodiments, a coating is Opadry® Purple.

In embodiments, a coating does not comprise polyvinyl alcohol (PVA) or polyvinylpyrrolidone (PVP) as a binder.

In embodiments, a tablet comprises:
an intragranular component comprising
   ferric citrate in an amount that is about 65-75 weight %;
   a binder in an amount that is about 3-8 weight %;
   a filler in an amount that is about 15-25 weight %;
   a disintegrant in an amount that is about 1-3 weight %; and
   a lubricant in an amount that is about 0.1-0.5 weight %; and
an extragranular component comprising
   one or more glidants in a total amount that is about 0.1-0.5 weight %; and
   one or more lubricants in a total amount that is about 0.3-0.8 weight %; and
an optional coating in an amount that is about 1-5 weight %, wherein said coating comprises a non-polyvinyl alcohol binder; and
wherein the weight % is determined based on the total weight of the tablet.

In embodiments, a tablet comprises:
an intragranular component comprising
   ferric citrate in an amount that is about 65-75 weight %;
   copovidone in an amount that is about 3-8 weight %;
   microcrystalline cellulose in an amount that is about 15-25 weight %;
   crospovidone in an amount that is about 1-3 weight %; and
   magnesium stearate in an amount that is about 0.1-0.5 weight %; and
an extragranular component comprising
   colloidal silicon dioxide in a total amount that is about 0.1-0.5 weight %; and
   magnesium stearate in a total amount that is about 0.3-0.8 weight %; and
an optional coating in an amount that is about 1-5 weight %, wherein said coating comprises a non-polyvinyl alcohol binder; and
wherein the weight % is determined based on the total weight of the tablet.

In embodiments, a tablet comprises:
an intragranular component comprising
   about 250 mg (±10% or ±5%) ferric citrate;
   about 17.9 mg (±10% or ±5%) copovidone;
   about 71.6 mg (±10% or ±5%) microcrystalline cellulose;
   about 7.1 mg (±10% or ±5%) crospovidone; and
   about 0.9 mg (±10% or ±5%) magnesium stearate; and
an extragranular component comprising
   about 0.7 mg (±10% or ±5%) colloidal silicon dioxide; and
   about 1.8 mg (±10% or ±5%) magnesium stearate; and
   about 14.0 g (±10% or ±5%) of a coating, wherein said coating comprises a non-polyvinyl alcohol binder.

In embodiments, a tablet coating comprises hydroxypropyl methylcellulose (HPMC) as the binder.

In embodiments, a tablet coating is Opadry® Purple.

In embodiments, a tablet is formulated for immediate release of the ferric citrate.

In embodiments, a tablet has a total weight of about 200-500 mg, 250-450 mg, or 300-400 mg.

In embodiments, a tablet has a hardness of about 10-20 or 12-18 kp.

In embodiments, a tablet has a friability that is ≤ about 1%.

In embodiments, a tablet has a disintegration time of ≤ about 20 or 15 minutes.

In embodiments, a tablet has a BET specific surface area greater than 5 m²/g.

In embodiments, a tablet has a BET specific surface area great than 10 m²/g.

In embodiments, a tablet has a BET specific surface area greater than 20 m²/g.

In embodiments, the BET specific surface area ranges from 20 m²/g to 40 m²/g, 25 m²/g to 35 m²/g, or 25 m²/g to 30 m²/g.

In embodiments, a pharmaceutical composition is formulated for administration as granules or a powder.

In another aspect, the present invention provides herein a method for the prophylaxis or treatment of hyperphosphatemia in a subject in need thereof, comprising administering to the subject an effective amount of ferric citrate, wherein the subject is 5 about 18 years of age, and wherein the subject has chronic kidney disease.

In embodiments, a subject is on dialysis.

In embodiments, a subject is of about 6-18 years of age.

In embodiments, a subject is about 6 to <18 years of age.

In embodiments, a subject receives a weight-based dose of ferric citrate.

In embodiments,
a subject of about 12 to <20 kg receives an initial daily dose of ferric citrate of about 1000 mg;
a subject of about 20 to <40 kg receives an initial daily dose of ferric citrate of about 2000 mg;
a subject of about 40 to <60 kg receives an initial daily dose of ferric citrate of about 3000 mg; or
a subject of about ≥60 kg receives an initial daily dose of ferric citrate of about 6000 mg.

In embodiments,
a subject of about 12 to <20 kg receives a maximum daily dose of ferric citrate of about 1000 mg, wherein the daily dose is optionally adjusted by increments of about 250 mg or about 1000 mg;
a subject of about 20 to <40 kg receives a maximum daily dose of ferric citrate of about 5000 mg, wherein the daily dose is optionally adjusted by increments of about 500 mg or about 2000 mg;
a subject of about 40 to <60 kg receives a maximum daily dose of ferric citrate of about 9000 mg, wherein the daily dose is optionally adjusted by increments of about 1000 mg or about 3000 mg; or
a subject of about ≥60 kg receives a maximum daily dose of ferric citrate of about 12000 mg, wherein the daily dose is optionally adjusted by increments of about 1000 mg or about 6000 mg.

In another aspect, the present invention provides herein a method of treating iron deficiency anemia in a subject in need thereof, comprising administering to the subject an effective amount of ferric citrate, wherein the subject is < about 18 years of age, and wherein the subject has chronic kidney disease.

In embodiments, a subject is not on dialysis.

In embodiments, a subject is about 6 to <18 years of age or about 12 to 17 years of age. In embodiments, a subject is about 6 to <18 years of age. In embodiments, a subject about 12 to 17 years of age.

In embodiments, a subject receives a weight-based dose of ferric citrate.

In embodiments,
a subject of about 12 to <40 kg receives an initial daily dose of about 750 mg ferric citrate;
a subject of about 40 to <60 kg receives an initial daily dose of about 1500 mg ferric citrate; or
a subject of about ≥60 kg receives an initial daily dose of about 3000 mg ferric citrate.

In embodiments,
a subject of about 12 to <40 kg receives a maximum daily dose of about 2250 mg ferric citrate, wherein the daily dose is optionally adjusted by increments of about 750 mg;
a subject of about 40 to <60 kg receives a maximum daily dose of about 4500 mg ferric citrate, wherein the daily dose is optionally adjusted by increments of about 1500 mg; or
a subject of about ≥60 kg receives a maximum daily dose of about 9000 mg ferric citrate, wherein the daily dose is optionally adjusted by increments of about 3000 mg.

In embodiments, a subject is about 12 to 17 years of age and/or about ≥40 kg.

In embodiments, ferric citrate is administered as a pharmaceutical composition.

In embodiments, a subject is of about 6-18 years of age or about 12 to 17 years of age.

In embodiments, a pharmaceutical composition is administered as a tablet.

In embodiments, a pharmaceutical composition described herein is to be administered.

In embodiments, provided herein are methods for preparing a pharmaceutical formulation as described herein, comprising a first step of blending the ferric citrate, the one or more binders, the one or more fillers, and the one or more disintegrants, of the intragranular phase to form a first pre-blend, and wherein the components are optionally screened prior to blending.

In embodiments, the method comprises blending the one or more lubricants of the intragranular phase with the first pre-blend to form the second pre-blend, wherein the one or more lubricants are optionally screened prior to blending.

In embodiments, the blended material is granulated by dry granulation process to form granules of suitable particle size distribution.

In embodiments, the method comprises a second step of blending the granules with the one or more glidants and the one or more lubricants of the extragranular component to form the blend, optionally wherein the one or more glidants and the one or more lubricants are screened prior to blending.

In embodiments, the blend is compressed to form a tablet.

In embodiments, compressed tablets are coated with suitable coating material consisting of cellulosic product.

In embodiments, a tablet comprises a coating, where the coating comprises hydroxypropylmethyl cellulose (HPMC) (e.g., as a binding agent).

### BRIEF DESCRIPTION OF THE DRAWINGS

FIG. 1 shows an exemplary manufacturing process suitable for preparing tablet compositions described herein.

FIG. 2A shows the mean dissolution profile of the coated granules with batch #118070-33 and batch #118070-36.

FIG. 2B shows the individual dissolution profile of the coated granules with batch #118070-85.

FIG. 3 illustrates the dissolution results of film-coated Ferric Citrate 250 mg Tablets having a HPMC- or PVA-based coating material under accelerated storage conditions (n=6).

### DETAILED DESCRIPTION

Disclosed herein are ferric citrate-containing tablets. In various embodiments, the tablets include ferric citrate formulations that meet certain dissolution, tableting and disintegration standards. In various aspects, the tablet formulations can include ferric citrate as the active ingredient and one or more excipients (e.g., binders, disintegrants, fillers, lubricants, or glidants).

Compositions described herein can be particularly beneficial for the treatment of pediatric patients.

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference for all purposes.

Animal: As used herein, the term “animal” refers to any member of the animal kingdom. In some embodiments, “animal” refers to humans, at any stage of development. In some embodiments, “animal” refers to non-human animals, at any stage of development. In embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Dose(s): As used herein, the term “dose(s)” means a quantity of the compound or a pharmaceutically acceptable salt, solvate, or hydrate thereof to be administered at one time. A dose may comprise a single unit dosage form, or alternatively may comprise more than a single unit dosage form (e.g., a single dose may comprise two tablets), or even less than a single unit dosage form (e.g., a single dose may comprise half of a tablet).

Daily dose: As used herein, the term “daily dose” means a quantity of the compound, or a pharmaceutically acceptable salt, solvate, or hydrate thereof that is administered in a 24-hour period. Accordingly, a daily dose may be administered all at once (i.e., once daily dosing) or alternatively the daily dosing may be divided such that administration of the compound is twice daily, three times daily, or even four times daily.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control sample or subject (or multiple control samples or subjects) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Pharmaceutically acceptable: The term “pharmaceutically acceptable”, as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quaternization of an amine using an appropriate electrophile, e.g., an alkyl halide, to form a quarternized alkylated amino salt.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one-unit dose.

Preventing: The term “prevent,” “preventing,” or “prevention,” as used herein refers to an effect that mitigates an undesired effect, e.g., an undesirable drug-drug interaction or the formation of a drug-iron chelate. Prevention does not require the 100% elimination of the possibility of an event. Rather, it denotes that the likelihood of the occurrence of the event has been reduced by the compound or method.

Treating: As used herein, the term “treat,” “treatment,” or “treating” refers to any method used to partially or completely alleviate, ameliorate, relieve, inhibit, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

As used herein, the term “unit dosage form(s)” includes tablets; caplets; capsules, such as soft elastic gelatin capsules; sachets; cachets; troches; lozenges; dispersions; powders; solutions; gels; liquid dosage forms suitable for oral or mucosal administration to a patient, including suspensions (e.g., aqueous or non-aqueous liquid suspensions), emulsions (e.g., oil-in-water emulsions, or a water-in-oil liquid emulsion), solutions, and elixirs; and sterile solids (e.g., crystalline or amorphous solids) that can be reconstituted to provide liquid dosage forms suitable for oral or parenteral administration to a patient.

The unit dosage form does not necessarily have to be administered as a single dose nor does a single unit dosage form necessarily constitute an entire dose.

Patient Populations

The ferric citrate or a pharmaceutical composition thereof disclosed herein may be administered to a subject in need thereof for treating a disease or condition described herein. For example, compositions described herein can be beneficial for pediatric subjects in need of treatment.

In embodiments, a subject is an adult. In embodiments, a subject is > about 18 years old.

In embodiments, a subject is a pediatric subject (e.g., a subject is ≤ about 18 years old). In embodiments, a subject is ≥ about 6 years old. In embodiments, a subject is about 6-18, 6-12, or 12-18 years of age. In embodiments, a subject is about 6, 7, 8, 9, 10, 11, 12, 13, 14, 15, 16, 17, or 18 years of age. In embodiments, a subject is no more than about 6-18, 6-12, or 12-18 years of age. In embodiments, a subject is no more than about 6, 7, 8, 9, 10, 11, 12, 13, 14, 15, 16, 17, or 18 years of age.

In embodiments, a subject has a disease or condition that benefits from administration of ferric citrate (e.g., formulations of ferric citrate as described herein). Exemplary methods of treatment are described herein.

In embodiments, a subject has chronic kidney disease (CKD). Individuals with a glomerular filtration rate (GFR)<60 ml/min/1.73 m² for 3 months or more can be classified as having CKD, irrespective of the presence or absence of kidney damage. In embodiments, the chronic kidney disease is stage 3, 4, or 5 chronic kidney disease. In certain embodiments, the chronic kidney disease is pre-dialysis chronic kidney disease. In other embodiments, the chronic kidney disease is non-dialysis dependent chronic kidney disease.

As described herein, methods can be useful for the treatment of subjects of having various dialysis statuses (e.g., a dialysis status as described herein).

In embodiments, the subject is non-dialysis dependent. For example, in some embodiments, the subject has non-dialysis chronic kidney disease (an NDD-CKD patient).

In embodiments, the subject is dialysis-dependent. For example, in embodiments, the subject has dialysis dependent chronic kidney disease (a DD-CKD patient).

In embodiments, the subject is a dialysis patient and these patients may be referred to as having end stage renal disease (ESRD).

In embodiments, the subject receives or previously has received dialysis. In embodiments, the subject receives dialysis. In embodiments, the patient previously received dialysis.

In embodiments, dialysis is hemodialysis (HD). In embodiments, the subject with chronic kidney disease receives or previously received hemodialysis. In embodiments, the subject with chronic kidney disease receives hemodialysis. In embodiments, the subject with chronic kidney disease previously received hemodialysis.

In embodiments, dialysis is peritoneal dialysis (PD). In embodiments, the subject with chronic kidney disease receives or previously received peritoneal dialysis. In embodiments, the subject with chronic kidney disease receives peritoneal dialysis. In embodiments, the subject with chronic kidney disease previously received peritoneal dialysis.

Pediatric Formulations (Pharmaceutical Compositions) of Ferric Citrate

In one aspect, provided herein are formulations (pharmaceutical compositions) comprising ferric citrate. In embodiments, a pharmaceutical composition comprising ferric citrate is formulated as a solid oral dosage form (e.g., granules, powders, tablets, capsules, and caplets). In embodiments, a pharmaceutical composition comprising ferric citrate is formulated as a tablet. In embodiments, a pharmaceutical composition comprising ferric citrate is formulated as granules or powders. Such pharmaceutical compositions contain predetermined amounts of active ingredients, and may be prepared by methods of pharmacy well known to those skilled in the art.

In embodiments, a pharmaceutical composition (e.g., a tablet) provided herein comprises ferric citrate. In embodiments, a pharmaceutical composition (e.g., a tablet) provided herein can further comprise one or more excipients. Suitable excipients are well known to those skilled in the art of pharmacy, and non-limiting examples of suitable excipients are provided herein. For example, excipients suitable for use in solid oral dosage forms (e.g., granules, powders, tablets, capsules, and caplets) include, but are not limited to, binders, disintegrants, fillers, lubricants, and glidants.

In embodiments, provided herein are pharmaceutical compositions (e.g., tablets) comprising intragranular components and extragranular components. In embodiments, an intragranular component comprises ferric citrate, one or more binders, one or more disintegrants, one or more fillers, and one or more lubricants. In embodiments, an extragranular component comprises one or more glidants and one or more lubricants. In embodiments, a pharmaceutical composition (e.g., a tablet) can further comprise film coating components.

In embodiments, provided herein are pharmaceutical compositions (e.g., tablets) comprising intra-granular components and extra-granular components, wherein the intragranular component comprises about 60-80 weight % by weight of ferric citrate, about 1-5 weight % by weight of disintegrants, about 10-30 weight % by weight of fillers, and about 0.1-2 weight % by weight of lubricants; wherein the extragranular component comprises about 0.1-2 weight % by weight of glidants, about 0.1-2 weight % by weight of lubricants; and wherein the weight % is determined based on the sum weights of the intragranular and extragranular components.

In embodiments, provided herein are pharmaceutical compositions (e.g., tablets) comprising an intra-granular component comprising ferric citrate present in an amount that is about 60-80 weight %; two or more excipients selected from the group consisting of copovidone, microcrystalline cellulose, and crospovidone, wherein said excipients are present in a total amount that is about 20-35 weight %; and magnesium stearate or calcium stearate present in an amount that is about 0.1-2 weight %; and an extragranular component comprising hydrophilic fumed silica or colloidal silicon dioxide present in an amount that is about 0.1-2 weight %; and magnesium stearate or calcium stearate present in an amount that is about 0.1-2 weight %; and wherein the weight % is determined based on the sum weights of the intragranular and extragranular components.

In embodiments, an intragranular component of a pharmaceutical composition (e.g., a tablet) comprises copovidone, microcrystalline cellulose, and crospovidone.

Ferric Citrate

In embodiments, a pharmaceutical composition (e.g., a tablet) described herein comprises ferric citrate. In embodiments, a pharmaceutical composition (e.g., a tablet) described herein comprises an intragranular component that comprises ferric citrate.

Ferric citrate is commercially available or may be prepared according to WO 2004/074444; WO 2007/022435; WO 2011/011541; and/or US20120121703, each of which is incorporated in its entirety.

In embodiments, the ferric citrate used as described herein is known chemically as iron (+3), x (1, 2, 3-propanetricarboxylic acid, 2-hydroxy-), y (H₂O)

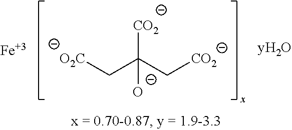

In embodiments, provided herein are pharmaceutical compositions (e.g., tablets) that comprise about 50 weight %, about 55 weight %, about 60 weight %, about 65 weight %, about 70 weight %, about 75 weight %, about 80 weight %, about 85 weight %, about 90 weight %, or about 95 weight % by weight of ferric citrate, wherein the weight % is determined based on the sum weights of the intragranular and extragranular components. In embodiments, provided herein are pharmaceutical compositions that comprise about 50-95 weight %, about 55-85 weight %, about 60-80 weight %, about 60-75 weight %, about 65-80 weight %, about 65-75 weight %, about 70-80 weight %, or about 70-75 weight %, by weight of ferric citrate, wherein the weight % is determined based on the sum weights of the intragranular and extragranular components. In embodiments, provided herein are pharmaceutical compositions that comprise about 65-75 weight % or about 70-75 weight %, by weight of ferric citrate, wherein the weight % is determined based on the sum weights of the intragranular and extragranular components.

In embodiments, provided herein are pharmaceutical compositions (e.g., tablets) that comprise about 100-1200 mg, 100-1100 mg, 100-1000 mg, 100-900 mg, 100-800 mg, 100-700 mg, 100-600 mg, 100-500 mg, 100-400 mg, 100-300 mg, 100-200 mg, 200-900 mg, 200-800 mg, 200-700 mg, 200-600 mg, 200-500 mg, 200-400 mg, 200-300 mg, 300-900 mg, 400-800 mg, 400-700 mg, 400-600 mg, 400-500 mg, 500-900 mg, 500-800 mg, 500-700 mg, or 500-600 mg ferric citrate. In embodiments, provided herein are pharmaceutical compositions (e.g., tablets) that comprise about 100-500 mg, 200-500 mg, or 300-500 mg ferric citrate.

In embodiments, provided herein are pharmaceutical compositions (e.g., tablets) that comprise about 50, 100, 150, 200, 250, 300, 350, 400, 450, 500, 550, 600, 650, 700, 750, 800, 850, 900, 950, 1000 mg, 1100 mg, or 1200 mg ferric citrate. In embodiments, provided herein are pharmaceutical compositions (e.g., tablets) that comprise about 50, 100, 150, 200, 250, 300, 350, 400, 450, or 500 mg ferric citrate. In embodiments, provided herein are pharmaceutical compositions (e.g., tablets) that comprise about 250 mg ferric citrate.

Binders

In embodiments, a pharmaceutical composition (e.g., a tablet) described herein comprises binders. In embodiments, a pharmaceutical composition (e.g., a tablet) described herein comprises an intragranular component that comprises binders.

In embodiments, a binder suitable for use in the pharmaceutical composition can be any binder known in the art. Without limitation, examples of the binder can include one or more of hydroxypropyl cellulose (HPC), hydroxypropylmethyl cellulose (HPMC), sodium alginate, alginic acid, guar gum, acacia gum, xanthan gum, carbolpol, cellulose gum (carboxy methyl cellulose), ethyl cellulose, maltodextrin, PVP/VA, povidone, microcrystalline cellulose, starch (partially or fully pregelatinized starch), methyl cellulose, or copovidone. The maltodextrin, PVP/VA, and methyl cellulose can function as immediate release binders when used in the ferric citrate formulations.

It also should be understood that combinations of binders can be used to control and vary the effect of the binder. For example, a binder system can be made up of hydroxypropyl cellulose and polyvinyl pyrrolidone (povidone) with or without microcrystalline cellulose. One or both of the hydroxypropyl cellulose and povidone can be replaced with pregelatinized starch.

In embodiments, a pharmaceutical composition (e.g., an intragranular component of a pharmaceutical composition) comprises a binder that is copovidone.

In embodiments, provided herein are pharmaceutical compositions (e.g., tablets) that comprise about 1 weight %, about 2 weight %, about 3 weight %, about 4 weight %, about 5 weight %, about 6 weight %, about 7 weight %, about 8 weight %, about 9 weight %, about 10 weight %, about 11 weight %, about 12 weight %, about 13 weight %, about 14 weight %, about 15 weight %, about 16 weight %, about 17 weight %, about 18 weight %, about 19 weight %, or about 20 weight % by weight of binders, wherein the weight % is determined based on the sum weights of the intragranular and extragranular components. In embodiments, provided herein are pharmaceutical compositions (e.g., tablets) that comprise about 1-20 weight %, about 1-15 weight %, about 1-10 weight %, about 3-10 weight %, about 3-9 weight %, about 3-8 weight %, about 3-7 weight %, about 3-6 weight %, about 3-5 weight %, about 4-10 weight %, about 4-9 weight %, about 4-8 weight %, about 4-7 weight %, or about 4-6 weight % by weight of binders, wherein the weight % is determined based on the sum weights of the intragranular and extragranular components. In embodiments, provided herein are pharmaceutical compositions (e.g., tablets) that comprise about 3-10 weight %, about 3-9 weight %, about 3-8 weight %, about 3-7 weight %, about 3-6 weight %, about 3-5 weight %, about 4-10 weight %, about 4-9 weight %, about 4-8 weight %, about 4-7 weight %, or about 4-6 weight % by weight of binders, wherein the weight % is determined based on the sum weights of the intragranular and extragranular components.

In embodiments, a pharmaceutical composition comprises about 1-10 weight % (e.g., about 4-6 weight % of a binder (e.g., copovidone).

Disintegrants

In embodiments, a pharmaceutical composition (e.g., a tablet) described herein comprises disintegrants. In embodiments, a pharmaceutical composition (e.g., a tablet) described herein comprises an intragranular component that comprises disintegrants.

In embodiments, a disintegrant can be the same as or different from a binder. By way of example and not limitation, microcrystalline cellulose has both binder and disintegrant properties and microcrystalline cellulose can be use as the sole binder/disintegrant in the formulation. Examples of other suitable disintegrants include, but are not limited to, croscarmellose sodium, crospovidone, sodium starch glycolate, starch, and mixtures thereof.

In embodiments, a pharmaceutical composition (e.g., an intragranular component of a pharmaceutical composition) comprises a disintegrant that is crospovidone.

In embodiments, provided herein are pharmaceutical compositions (e.g., tablets) that comprise about 1 weight %, about 1.5 weight %, about 2 weight %, about 2.5 weight %, about 3 weight %, about 3.5 weight %, about 4 weight %, about 4.5 weight %, about 5 weight %, about 5.5 weight %, about 6 weight %, about 6.5 weight %, about 7 weight %, about 7.5 weight %, about 8 weight %, about 8.5 weight %, about 9 weight %, about 9.5 weight %, or about 10 weight % by weight of disintegrants, wherein the weight % is determined based on the sum weights of the intragranular and extragranular components. In embodiments, provided herein are pharmaceutical compositions (e.g., tablets) that comprise about 1-10 weight %, about 1-8 weight %, about 1-5 weight %, about 1-4.5 weight %, about 1-4 weight %, about 1-3.5 weight %, about 1-3 weight %, about 2-5 weight %, about 2-4.5 weight %, about 2-4 weight %, about 2-3.5 weight %, about 1-2 weight %, about 2-3 weight %, about 3-4 weight %, or about 4-5 weight % by weight of disintegrants, wherein the weight % is determined based on the sum weights of the intragranular and extragranular components. In embodiments, provided herein are pharmaceutical compositions (e.g., tablets) that comprise about 1-2 weight %, about 2-3 weight %, about 3-4 weight %, or about 4-5 weight % by weight of disintegrants, wherein the weight % is determined based on the sum weights of the intragranular and extragranular components.

In embodiments, a pharmaceutical composition comprises about 1-5 weight % (e.g., about 1-2 or 2-3 weight % of a disintegrant (e.g., crospovidone)

Fillers

In embodiments, a pharmaceutical composition (e.g., a tablet) described herein comprises fillers. In embodiments, a pharmaceutical composition (e.g., a tablet) described herein comprises an intragranular component that comprises fillers.

Examples of fillers suitable for use in the pharmaceutical compositions include, but are not limited to, microcrystalline cellulose, starches, partially pregelatinized starches, sorbitol powder, mannitol powder, lactose, hydroxypropyl methyl cellulose, hydroxypropyl cellulose, maltodextrins, dried glucose syrup, dextrose mono & anhydrous, and mixtures thereof.

Other suitable fillers include, but are not limited to, talc, calcium carbonate (e.g., granules or powder), powdered cellulose, dextrates, kaolin, mannitol, silicic acid, sorbitol, pre-gelatinized starch, and mixtures thereof.

In embodiments, fillers may include, but are not limited to block copolymers of ethylene oxide and propylene oxide. Such block copolymers may be sold as POLOXAMER or PLURONIC, and include, but are not limited to POLOXAMER 188 NF, POLOXAMER 237 NF, POLOXAMER 338 NF, POLOXAMER 437 NF, and mixtures thereof.

In embodiments, fillers may include, but are not limited to isomalt, lactose, lactitol, mannitol, sorbitol xylitol, erythritol, and mixtures thereof.

In embodiments, a pharmaceutical composition (e.g., an intragranular component of a pharmaceutical composition) comprises a filler that is microcrystalline cellulose.

In embodiments, provided herein are pharmaceutical compositions (e.g., tablets) that comprise about 2 weight %, about 4 weight %, about 6 weight %, about 8 weight %, about 10 weight %, about 12 weight %, about 14 weight %, about 16 weight %, about 18 weight %, about 20 weight %, about 22 weight %, about 24 weight %, about 26 weight %, about 28 weight %, about 30 weight %, about 32 weight %, about 34 weight %, about 36 weight %, about 38 weight %, or about 40 weight % by weight of fillers, wherein the weight % is determined based on the sum weights of the intragranular and extragranular components. In embodiments, provided herein are pharmaceutical compositions (e.g., tablets) that comprise about 1-40 weight %, 1-35 weight %, 1-30 weight %, 1-25 weight %, 1-20 weight %, about 5-40 weight %, about 5-35 weight %, about 5-30 weight %, about 5-25 weight %, about 5-20 weight %, about 10-40 weight %, about 10-35 weight %, about 10-30 weight %, about 10-25 weight %, about 10-20 weight %, about 15-40 weight %, about 15-35 weight %, about 15-30 weight %, about 15-25 weight %, about 15-20 weight %, about 20-40 weight %, about 20-35 weight %, about 20-30 weight %, about 20-25 weight %, about 25-40 weight %, about 25-35 weight %, or about 25-30 weight % by weight of fillers, wherein the weight % is determined based on the sum weights of the intragranular and extragranular components. In embodiments, provided herein are pharmaceutical compositions (e.g., tablets) that comprise about 10-25 weight %, 10-20 weight %, 15-25 weight %, 15-30 weight %, 20-30 weight %, or about 20-25 weight % by weight of fillers, wherein the weight % is determined based on the sum weights of the intragranular and extragranular components.

In embodiments, a pharmaceutical composition comprises about 10-30 weight % (e.g., about 15-25 or 20-25 weight % of a filler (e.g., microcrystalline cellulose)

Lubricants

In embodiments, a pharmaceutical composition (e.g., a tablet) described herein comprises lubricants. In embodiments, a pharmaceutical composition (e.g., a tablet) described herein comprises an intragranular component that comprises lubricants. In embodiments, a pharmaceutical composition (e.g., a tablet) described herein comprises an extragranular component that comprises lubricants.

In embodiments, an intragranular component comprises one or more lubricants. In embodiments, an extragranular component comprises one or more lubricants. Examples of lubricants suitable for use in the pharmaceutical compositions (e.g., in an intragranular component and/or an extragranular component) include, but are not limited to, magnesium stearate, calcium stearate, sodium stearyl fumarate, and mixtures thereof. Other suitable lubricants include one or more of polyethylene glycol (e.g., molecular weight above 3350), sodium lauryl sulfate, talc, mineral oil, leucine, and poloxamer.

In embodiments, the one or more lubricants comprised in an intragranular component and one or more lubricants comprised in an extragranular component are the same. In embodiments, the one or more lubricants comprised in an intragranular component and one or more lubricants comprised in an extragranular component are different.

In embodiments, a pharmaceutical composition (e.g., an intragranular component and/or in an extragranular component of a pharmaceutical composition) comprises a lubricant that is magnesium stearate. In embodiments, a pharmaceutical composition (e.g., an intragranular component and/or in an extragranular component of a pharmaceutical composition) comprises a lubricant that is calcium stearate.

In embodiments, provided herein are pharmaceutical compositions (e.g., tablets) that comprise about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2 weight %, about 2.2 weight % about 2.4 weight % about 2.6 weight % about 2.8 weight % about 3 weight % about 3.2 weight %, about 3.4 weight %, about 3.6 weight %, about 3.8 weight %, or about 4 weight % by weight of lubricants, wherein the weight % is determined based on the sum weights of the intragranular and extragranular components. In embodiments, provided herein are pharmaceutical compositions (e.g., tablets) that comprise about 0.1-4 weight %, about 0.1-3.5 weight %, about 0.1-3 weight %, about 0.1-2.5 weight %, about 0.1-2 weight %, about 0.1-1.5 weight %, about 0.1-1 weight %, about 0.1-0.5 weight %, about 0.5-2 weight %, about 0.5-1.5 weight %, or about 0.5-1 weight %, by weight of lubricants, wherein the weight % is determined based on the sum weights of the intragranular and extragranular components. In embodiments, provided herein are pharmaceutical compositions (e.g., tablets) that comprise about 0.1-1 weight % by weight of lubricants, wherein the weight % is determined based on the sum weights of the intragranular and extragranular components.

In embodiments, one or more lubricants of the intragranular component are present in a total amount of about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2 weight %, about 2.2 weight % about 2.4 weight % about 2.6 weight % about 2.8 weight % about 3 weight % about 3.2 weight %, about 3.4 weight %, about 3.6 weight %, about 3.8 weight %, or about 4 weight %. In embodiments, one or more lubricants of the intragranular component are present in a total amount of about 0.1-4 weight %, about 0.1-3.5 weight %, about 0.1-3 weight %, about 0.1-2.5 weight %, about 0.1-2 weight %, about 0.1-1.5 weight %, about 0.1-1 weight %, about 0.1-0.5 weight %, about 0.5-2 weight %, about 0.5-1.5 weight %, or about 0.5-1 weight %. In embodiments, one or more lubricants of the intragranular component are present in a total amount of about 0.1-1 weight %.

In embodiments, one or more lubricants of the extragranular component are present in a total amount of about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2 weight %, about 2.2 weight % about 2.4 weight % about 2.6 weight % about 2.8 weight % about 3 weight % about 3.2 weight %, about 3.4 weight %, about 3.6 weight %, about 3.8 weight %, or about 4 weight %. In embodiments, one or more lubricants of the extragranular component are present in a total amount of about 0.1-4 weight %, about 0.1-3.5 weight %, about 0.1-3 weight %, about 0.1-2.5 weight %, about 0.1-2 weight %, about 0.1-1.5 weight %, about 0.1-1 weight %, about 0.1-0.5 weight %, about 0.5-2 weight %, about 0.5-1.5 weight %, or about 0.5-1 weight %. In embodiments, one or more lubricants of the extragranular component are present in a total amount of about 0.1-1 weight %.

In embodiments, a pharmaceutical composition comprises about 0.1-2 weight % (e.g., about 0.1-1 or 0.1-0.5 weight %) of a lubricant (e.g., magnesium stearate).

Glidants

In embodiments, a pharmaceutical composition (e.g., a tablet) described herein comprises glidants. In embodiments, a pharmaceutical composition (e.g., a tablet) described herein comprises an extragranular component that comprises lubricants.

Examples of glidants suitable for use in the pharmaceutical compositions include, but are not limited to, hydrophilic fumed silica, colloidal silicon dioxide, starch, talc, magnesium stearate, and mixtures thereof.

In embodiments, a suitable glidant is colloidal silicon dioxide.

In embodiments, a suitable glidant is hydrophilic fumed silica having a BET specific surface area ranges from 50 to 400 m²/g. In embodiments, a suitable glidant is hydrophilic fumed silica having a BET specific surface area ranges from 50 to 100 m²/g, 50 to 200 m²/g, 50 to 300 m²/g, 50 to 400 m²/g, 100 to 200 m²/g, 100 to 300 m²/g, 100 to 400 m²/g, 200 to 300 m²/g, 200 to 400 m²/g, or 300 to 400 m²/g. In embodiments, a suitable glidant is hydrophilic fumed silica having a BET specific surface area of about 50 m²/g, about 60 m²/g, about 70 m²/g, about 80 m²/g, about 90 m²/g, about 100 m²/g, about 110 m²/g, about 120 m²/g, about 130 m²/g, about 140 m²/g, about 150 m²/g, about 160 m²/g, about 170 m²/g, about 180 m²/g, about 190 m²/g, about 200 m²/g, about 210 m²/g, about 220 m²/g, about 230 m²/g, about 240 m²/g, about 250 m²/g, about 260 m²/g, about 270 m²/g, about 280 m²/g, about 290 m²/g, about 300 m²/g, about 310 m²/g, about 320 m²/g, about 330 m²/g, about 340 m²/g, about 350 m²/g, about 360 m²/g, about 370 m²/g, about 380 m²/g, about 390 m²/g, or about 400 m²/g. In embodiments, a suitable glidant is hydrophilic fumed silica having a BET specific surface area of about 50 m²/g, about 100 m²/g, about 200 m²/g, about 300 m²/g, or about 400 m²/g.

Other suitable glidants include, but are not limited to, calcium stearate, mineral oil, light mineral oil, glycerin, sorbitol, mannitol, polyethylene glycol, other glycols, stearic acid, sodium stearyl fumarate, sodium lauryl sulfate, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional glidants include, for example, a syloid silica gel (e.g., Aerosil 200), a coagulated aerosol of synthetic silica, and mixtures thereof.

In embodiments, a pharmaceutical composition (e.g., an extragranular component of a pharmaceutical composition) comprises a glidant that is colloidal silicon dioxide.

In embodiments, a pharmaceutical composition (e.g., an extragranular component of a pharmaceutical composition) comprises a glidant that is hydrophilic fumed silica.

In embodiments, provided herein are pharmaceutical compositions (e.g., tablets) that comprise about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2 weight %, about 2.2 weight % about 2.4 weight % about 2.6 weight % about 2.8 weight % about 3 weight % about 3.2 weight %, about 3.4 weight %, about 3.6 weight %, about 3.8 weight %, or about 4 weight % by weight of glidants, wherein the weight % is determined based on the sum weights of the intragranular and extragranular components. In embodiments, provided herein are pharmaceutical compositions (e.g., tablets) that comprise about 0.1-4 weight %, about 0.1-3.5 weight %, about 0.1-3 weight %, about 0.1-2.5 weight %, about 0.1-2 weight %, about 0.1-1.5 weight %, about 0.1-1 weight %, about 0.1-0.5 weight %, about 0.5-2 weight %, about 0.5-1.5 weight %, or about 0.5-1 weight %, by weight of glidants, wherein the weight % is determined based on the sum weights of the intragranular and extragranular components. In embodiments, one or more glidants are present in a total amount of about 0.1-1 weight %.

In embodiments, a pharmaceutical composition comprises about 0.1-2 weight % (e.g., about 0.1-0.5 weight % of a glidant (e.g., colloidal silicon dioxide or hydrophilic fumed silica).

Tablet Coating

In embodiments, a tablet described herein can comprise a coating. For example, tablets described herein may comprise a coating that is suitable for achieving a desired pharmacokinetic profile (e.g., a favorable dissolution profile suitable for immediate release of ferric citrate). Such a desired pharmacokinetic profile may result from selection of a coating that comprises a suitable polymeric binding agent.

In embodiments, tablets are coated with suitable coating material consisting of cellulosic product. In embodiments, tablets are coated to a weight gain of approximately 1% to 10%, or 1% to 5%.

In embodiments, a tablet is coated using an Opadry® suspension or equivalent in a perforated pan coater. In embodiments, calcium stearate and Opadry® purple can be replaced with or used with a different lubricant or coating system, respectively.

In embodiments, a coating comprises hydroxypropylmethyl cellulose (HPMC) as a binding agent. In embodiments, a coating comprises hydroxypropylmethyl cellulose (HPMC) as the sole binding agent. In embodiments, a coating comprises hydroxypropylmethyl cellulose (HPMC) in an amount that is at least about 5, 10, 15, 20, 25, 30, 35, 40, 45, 50, 55, or 60% (w/w) of the total components of the coating. In embodiments, a coating is Opadry®Purple.

The trademark “Opadry®” is well-understood in the art and refers to a film coating product (e.g., a film coating system combining polymer, plasticizer and pigment). Exemplary Opadry® coatings include Opadry®Purple and Opadry®QX Pink. In embodiments, a coating is Opadry®Purple. In embodiments, a coating is Opadry®QX Pink.

Exemplary Opadry®formulations are provided in Table A.

TABLE A
Opadry ® Purple and Opadry ® QX Pink Coatings

| Opadry ® Purple | | Opadry ® QX Pink | | |
|---|---|---|---|---|
| | Quantities | | Quantities | |
| Component | % w/w | Component | % w/w | Function |
| Hypromellose | 62.5 | Macrogol (PEG) | 40.0 | Binding |
| (USP/Ph. | | Polyvinyl Alcohol | | polymer |
| Eur/JP) | | Graft Copolymer | | |
| | | (NF, Ph. Eur.) | | |
| | | Polyvinyl Alcohol | 3.5 | |
| | | (PVA)-Partially | | |
| | | Hydrolyzed | | |
| | | (USP, Ph. Eur.) | | |
| — | — | Talc (USP, Ph. | 27.5 | Anti- |
| | | Eur.) | | tacking |
| | | | | agent |
| Triacetin | 12.5% | GMCC Type 1 (NF, | 4.0 | Plasticizer |
| (USP/FCC/Ph. | | Ph. Eur.)/Mono/Di- | | |
| Eur./JPE) | | Glycerides (FCC) | | |
| Titanium | 21.8% | Titanium Dioxide | 21.8 | Opacifier |
| Dioxide | | (USP, Ph. Eur.) | | |
| (USP/NF/FCC/ | | | | |
| JP/Ph. Eur.) | | | | |
| FD&C Yellow | 1.73 | FD&C Yellow #6/ | 0.300 | Colorant |
| #6/Sunset | | Sunset Yellow FCF | | |
| Yellow FCF | | Aluminum Lake | | |
| Aluminum | | | | |
| Lake | | | | |
| FD&C Red | 1.19 | FD&C Red #40/ | 0.150 | |
| #40/Allura Red | | Allura Red AC | | |
| AC Aluminum | | Aluminum Lake | | |
| Lake | | | | |
| FD&C Blue | 0.32 | FD&C Blue #2/ | 0.100 | |
| #2/Indigo | | Indigo Carmine | | |
| Carmine | | Aluminum Lake | | |
| Aluminum Lake | | | | |

In embodiments, a tablet comprising a coating that comprises a HPMC binder is suitable for tablets formulated for immediate release of the ferric citrate (Q=80% in less than 60 minutes). In embodiments, such coated tablets also demonstrate suitable stability under accelerated storage conditions.

Tablet Weight

The weight of individual tablets can depend upon the final dosage to be produced. In embodiments, a tablet has a total weight of about 100 mg to about 2000 mg, about 100 about 100 mg to about 1700 mg, about 100 about 100 mg to about 1500 mg, about 100 mg to about 1300 mg, about 100 mg to about 1000 mg, about 100 mg to about 800 mg, or about 100 mg to about 500 mg. In embodiments, a tablet has a total weight of about 200 mg to about 500 mg, about 250 mg to about 450 mg, or about 300 mg to about 400 mg.

Characteristics of the Ferric Citrate Tablets

In embodiments, a pharmaceutical composition comprising ferric citrate is formulated as a tablet. In embodiments, the ferric citrate tablets disclosed herein display an enhanced BET specific surface area. BET theory explains the physical adsorption of gas molecules onto a solid surface. The theory serves as the basis for the measurement of the specific surface area of a material. This theory allows the calculation of surface areas of materials in a very accurate manner and is thus capable of distinguishing differences between separate preparations of what would otherwise appear to be the same material.

In embodiments, a tablet disclosed herein has a BET specific surface area greater than 5 m²/g. In embodiments, a tablet disclosed herein has a BET specific surface area greater than 10 m²/g. In embodiments, a tablet disclosed herein has a BET specific surface area greater than 20 m²/g. In embodiments, a tablet disclosed herein has a BET specific surface area ranges from 5 m²/g to 40 m²/g, 10 m²/g to 40 m²/g, 20 m²/g to 40 m²/g, 30 m²/g to 40 m²/g, 5 m²/g to 30 m²/g, or 10 m²/g to 30 m²/g, 20 m²/g to 30 m²/g, 5 m²/g to 20 m²/g, 10 m²/g to 20 m²/g. In embodiments, a tablet disclosed herein has a BET specific surface area ranges from 20 m²/g to 40 m²/g, 25 m²/g to 35 m²/g, or 25 m²/g to 30 m²/g. In embodiments, a tablet disclosed herein has a BET specific surface area of about 5 m²/g, about 6 m²/g, about 7 m²/g, about 8 m²/g, about 9 m²/g, about 10 m²/g, about 11 m²/g, about 12 m²/g, about 13 m²/g, about 14 m²/g, about 15 m²/g, about 16 m²/g, about 17 m²/g, about 18 m²/g, about 19 m²/g, about 20 m²/g, about 21 m²/g, about 22 m²/g, about 23 m²/g, about 24 m²/g, about 25 m²/g, about 26 m²/g, about 27 m²/g, about 28 m²/g, about 29 m²/g, about 30 m²/g, about 31 m²/g, about 32 m²/g, about 33 m²/g, about 34 m²/g, about 35 m²/g, about 36 m²/g, about 37 m²/g, about 38 m²/g, about 39 m²/g, about 40 m²/g. In embodiments, tablet disclosed herein has a BET specific surface area of at least about 5 m²/g, about 6 m²/g, about 7 m²/g, about 8 m²/g, about 9 m²/g, about 10 m²/g, about 11 m²/g, about 12 m²/g, about 13 m²/g, about 14 m²/g, about 15 m²/g, about 16 m²/g, about 17 m²/g, about 18 m²/g, about 19 m²/g, about 20 m²/g, about 21 m²/g, about 22 m²/g, about 23 m²/g, about 24 m²/g, about 25 m²/g, about 26 m²/g, about 27 m²/g, about 28 m²/g, about 29 m²/g, about 30 m²/g, about 31 m²/g, about 32 m²/g, about 33 m²/g, about 34 m²/g, about 35 m²/g, about 36 m²/g, about 37 m²/g, about 38 m²/g, about 39 m²/g, about 40 m²/g.

In embodiments, a tablet described herein has a disintegration time of ≤ about 25, 20, 15, or 10 minutes. In embodiments, a tablet as described herein has a disintegration time of ≤ about 20 or 15 minutes. In embodiments, a tablet as described herein has a disintegration time of about 1, 2, 3, 4, 5, 6, 7, 8, 9, 10, 11, 12, 13, 14, 15, 16, 17, 18, 19, 20, 21, 22, 23, 24, or 25 minutes. In embodiments, a tablet as described herein has a disintegration time of no more than about 1, 2, 3, 4, 5, 6, 7, 8, 9, 10, 11, 12, 13, 14, 15, 16, 17, 18, 19, 20, 21, 22, 23, 24, or 25 minutes.

Friability generally measures the mechanical strength of tablets. During the process of coating, transportation, packing, and other processes, tablets can lose weight. To measure the weight loss the samples are counted and weighed.

In various embodiments, a friability test is performed as described in United States Pharmacopeia Compendium of Standards (2007), which is incorporated herein by reference in its entirety. In embodiments, the tablets can be tested following USP <1216> for friability.

In embodiments, a tablet described herein has a friability that is ≤ about 1%, 3% or 5%. In embodiments, a tablet described herein has a friability that is < about 1%.

In embodiments, the tablets can be tested according to USP <1217> for hardness/breaking strength.

In embodiments, a tablet described herein has a hardness of about 10-20 or 12-18 kp. In embodiments, a tablet as described herein has a hardness of about 10, 11, 12, 13, 14, 15, 16, 17, 18, 19, or 20 kp.

In embodiments, a tablet described herein has a dissolution profile suitable for immediate release of the active ingredient ferric citrate (e.g., Q=80% in less than 60 minutes).

Method of Making Tablets

A tablet comprising ferric citrate as described herein can be prepared according to methods known in the art. Exemplary methods for making tablets comprising ferric citrate are described in WO 2011/011541, which is incorporated by reference in its entirety. Still further exemplary formulations are described herein.

In embodiments, a method described herein comprises a step (e.g., a first step) of blending the ferric citrate, the one or more binders, the one or more fillers, and the one or more disintegrants, of the intragranular phase to form a first pre-blend. In embodiments, the components are optionally screened prior to blending.

In embodiments, the step (e.g., the first step) further comprises blending the one or more lubricants of the intragranular phase with the first pre-blend to form the second pre-blend. In embodiments, the one or more lubricants are optionally screened prior to blending.

In embodiments, blended material described herein is granulated by dry granulation process to form granules of suitable particle size distribution.

In embodiments, a method described herein comprises a step (e.g., a second step) of blending the granules with the one or more glidants and the one or more lubricants of the extragranular component to form the blend. In embodiments, the one or more glidants and the one or more lubricants are screened prior to blending.

In embodiments, the blend is compressed to form a tablet.

In embodiments, the compressed tablets are coated with suitable coating material (e.g., coating material consisting of cellulosic product).

### Methods of Treatment

Exemplary methods of treatment comprising administering to a subject in need thereof an effective amount of ferric citrate are described in e.g., WO 2007/089577, WO 2007/089571, WO 2011/011541, WO 2013/192565, WO 2016/141124, and U.S. Pat. No. 5,753,706, which are incorporated by reference in their entirety. Still further exemplary formulations are described herein.

Described herein are therapeutic methods comprising administering ferric citrate or a pharmaceutical composition (e.g., a tablet) thereof in subjects in need thereof. For example, methods described herein can be useful for treating a disease or condition such as hyperphosphatemia or iron deficiency anemia. In embodiments, the pharmaceutical composition disclosed herein is administered to any chronic kidney disease (CKD) patients to treat any of the conditions and disorders described herein.

When used herein, a “250 mg ferric citrate tablet” refers to tablets comprising about 250 mg ferric citrate where excipients (including any coating) will modify the total weight of the dosage form. Likewise, a “1000 mg ferric citrate tablet” refers to tablets comprising about 1000 mg ferric citrate where excipients (including any coating) will modify the total weight of the dosage form.

In embodiments, methods described herein comprise administration of a formulation of ferric citrate described herein (e.g., a pediatric formulation). In embodiments, a pediatric formulation comprises

In embodiments, methods described herein comprise administration of ferric citrate in an alternative formulation, including as described in WO 2011/011541. In embodiments, a method described herein comprises administration of a ferric citrate tablet comprising (1) a core comprising 80.0-90.0% by weight of ferric citrate, 8.0-15.0% by weight of pregelatinized starch, and 1.0-3.0% by weight of calcium stearate; and (2) a coating.

In embodiments, a method described herein comprises administration of an oral dosage form (e.g., a ferric citrate tablet) comprising about 250-1000 mg ferric citrate.

In embodiments a ferric citrate tablet comprises about 1000 mg ferric citrate.

In embodiments, a ferric citrate tablet comprises about 250 mg ferric citrate (e.g., according to any formulation described herein). In embodiments, the ferric citrate is administered as a coated tablet (e.g., a coated 250 mg ferric citrate tablet where the coating comprises a binding agent that is HPMC, including as described herein).

In embodiments, the ferric citrate is administered as a combination of tablets comprising 250 mg ferric citrate and 1000 mg ferric citrate.

Hyperphosphatemia

Hyperphosphatemia is an electrolyte disorder in which there is an elevated level of phosphate in the blood. Causes of hyperphosphatemia include kidney failure, pseudohypoparathyroidism, hypoparathyroidism, diabetic ketoacidosis, tumor lysis syndrome, and rhabdomyolysis. Diagnosis of hyperphosphatemia can be based on a blood phosphate levels of greater than 1.46 mmol/L (4.5 mg/dL). When levels are greater than 4.54 mmol/L (14 mg/dL), it can be deemed severe. Levels may appear falsely elevated with high blood lipid levels, high blood protein levels, or high blood bilirubin levels.

Ferric iron can bind dietary phosphate in the GI tract (Gastrointestinal tract) and precipitates as ferric phosphate, and can lower the phosphate concentration in the serum. In embodiments, ferric citrate can be used as a phosphate binder for controlling serum phosphorus levels in subjects in need thereof (e.g., in patients with CKD).

In embodiments, ferric citrate is administered to a subject to reduce and/or control serum phosphorus levels. In embodiments, ferric citrate is administered to a subject to increase serum bicarbonate levels. In embodiments, ferric citrate is administered to a subject to increase serum iron parameters, including ferritin, iron and transferrin saturation (TSAT).

In embodiments, the pharmaceutical composition disclosed herein can be administered to CKD patients. In embodiments, the formulation disclosed herein can be administered to CKD patients to reduce and/or control serum phosphorus. In embodiments, the formulation disclosed herein can be administered to CKD patients to increase serum bicarbonate levels. In embodiments, the formulation disclosed herein can be administered to CKD patients to increase serum iron parameters, including ferritin, iron and transferrin saturation (TSAT).

In embodiments, provided herein are methods for the prophylaxis or treatment of hyperphosphatemia in a subject in need thereof, comprising administering to the subject an effective amount of ferric citrate. In embodiments, a subject is ≤ about 18 years of age. In embodiments, a subject has chronic kidney disease.

In embodiments, a subject is a child of ages 6 to <18 years. In embodiments, a subject is a child of ages 12 to <17 years. In embodiments, a subject has hyperphosphatemia related to chronic kidney disease (CKD). In embodiments, a subject has hyperphosphatemia related to CKD for at least about three months prior to commencement of ferric citrate therapy. In embodiments, a subject is on dialysis (e.g., chronic dialysis). In embodiments, a subject has non-dialysis-dependent CKD (ND-CKD). In embodiments, a subject has dialysis-dependent CKD (DD-CKD). In embodiments, a subject of age 12 years has a serum phosphate level of about >5.8 mg/dl. In embodiments, a subject of about 13 to <17 years of age has a serum phosphorus level of about >4.5 mg/dl.

In embodiments, a subject receives ferric citrate as an oral dosage form (e.g., a tablet such as a coated tablet) comprising 1 g ferric citrate (210 mg of ferric iron). In embodiments, a subject receives ferric citrate as an oral dosage form (e.g., a tablet such as a coated tablet) comprising 250 mg ferric citrate (52.5 mg of ferric iron).

In embodiments, a method described herein can result in a beneficial change to one or more physiological parameters (e.g., Hgb, TSAT, ferritin, serum phosphorus, calcium, and/or bicarbonate). A beneficial change can be a change in any of the parameters (e.g., an increase or a decrease) from a baseline (e.g., prior to or at commencement of a therapy, including as described herein) in a direction that is closer to or at the desired and/or target range.

In embodiments, a method described herein results in a beneficial change from baseline in serum phosphorus.

In embodiments, administration of ferric citrate according to methods described herein results in a serum phosphorus level of 3.6 to 5.8 mg/dl in a subject of 26 to 513 years of age.

In embodiments, administration of ferric citrate according to methods described herein results in a serum phosphorus level of 2.3 to 4.5 mg/dl in a subject of ≥13 to <18 years of age.

In embodiments, a method comprises administration of an initial (starting) dose to a subject. In embodiments, a method comprises weight-based dosing for a subject. In embodiments, a method comprises administration of ferric citrate according to any individual feature (e.g., any amount or modification) described in Tables 6 and 7 described herein, or any combination thereof.

In embodiments, a subject of a weight that is about 12 kg to <20 kg receives an initial (starting) daily dose of about 1000 mg ferric citrate. In embodiments, a dose may be modified by increments of about 250-1000 mg (e.g., about 250 mg or about 1000 mg increments). In embodiments, a maximum daily dose is about 2500 mg. In embodiments, the ferric citrate is administered as 250 mg tablets (e.g., according to any formulation described herein).

In embodiments, a subject of a weight that is about 20 kg to <40 kg receives an initial (starting) daily dose of about 2000 mg ferric citrate. In embodiments, a dose may be modified by increments of about 250-2000 mg (e.g., about 250 mg, about 500 mg, about 1000 mg, or about 2000 mg increments). In embodiments, a maximum daily dose is about 5000 mg. In embodiments, the ferric citrate is administered as 250 mg tablets (e.g., according to any formulation described herein).

In embodiments, a subject of a weight that is about 40 kg to <60 kg receives an initial (starting) daily dose of about 3000 mg ferric citrate. In embodiments, a dose may be modified by increments of about 1000-3000 mg (e.g., about 1000 mg, about 2000 mg, or about 3000 mg increments). In embodiments, a maximum daily dose is about 9000 mg. In embodiments, the ferric citrate is administered as 1000 mg tablets (e.g., according to any formulation described herein).

In embodiments, a subject of a weight that is about; 60 kg receives an initial (starting) daily dose of about 6000 mg ferric citrate. In embodiments, a dose may be modified by increments of about 1000-6000 mg (e.g., about 1000 mg, about 2000 mg, about 3000 mg, about 4000 mg, about 5000 mg, or about 6000 mg increments). In embodiments, a maximum daily dose is about 12000 mg. In embodiments, the ferric citrate is administered as 1000 mg tablets (e.g., according to any formulation described herein).

In embodiments, a daily dose of ferric citrate is modified. In embodiments, a daily dose of ferric citrate is modified based on the serum phosphorus levels of the subject. In embodiments, a method comprises modification of the daily dose of ferric citrate according to any individual feature (e.g., any amount or modification) described in Tables 8 and 9 described herein, or any combination thereof.

In embodiments, a daily dose of ferric citrate is modified based on certain iron parameters.

In embodiments, if a patient has a transferrin saturation (TSAT) of 250% (e.g., in a repeat assessment such as a follow-up assessment), the daily dose of ferric citrate may be adjusted by about ⅓ of the total daily dose. In embodiments, a daily dose of ferric citrate is decreased by 250 mg/day for subjects 12 to <20 kg. In embodiments, a daily dose of ferric citrate is decreased by 500 mg/day for subjects 20 to <40 kg. In embodiments, a daily dose of ferric citrate is decreased by 1000 mg/day for subjects 40 to <60 kg. In embodiments, a daily dose of ferric citrate is decreased by 2000 mg/kg for subjects 260 kg.

In embodiments, ferric citrate is administered as monotherapy.

In embodiments, ferric citrate is administered in combination with another therapy.

In embodiments, ferric citrate is administered in combination with IV iron therapy (e.g., for subjects having a TSAT <30%). In embodiments, ferric citrate is administered in combination with an erythropoietin stimulating agent (ESA). In embodiments, ferric citrate is administered in combination with a vitamin D and/or calcium supplement.

In embodiments, ferric citrate is not administered in combination with certain other therapies (e.g., a second phosphate binder (e.g., an aluminium-containing phosphate binder), oral iron therapy, or commercial ferric citrate.

Iron Deficiency Anemia

Iron deficiency anemia (IDA) can be characterized by pallor (pale color resulting from reduced oxyhemoglobin in the skin and mucous membranes), fatigue, lightheadedness, and weakness. However, signs of IDA can vary among patients.

IDA can be caused by insufficient dietary intake of iron, insufficient absorption of iron, insufficient storage of iron, and/or iron loss from bleeding which can originate from a number of sources such as the gastrointestinal, uterine or urinary tract. Therefore, it is commonly associated with conditions and disorders such as acute blood loss, chronic blood loss, childbirth, menstruation, gastrointestinal disorders (e.g., inflammatory bowel disease (IBD)), Chronic Kidney Disease (CKD), parasitic infections, insufficient dietary intake of iron, and insufficient absorption of iron.

In embodiments, ferric citrate can be an iron replacement product indicated for the treatment of iron deficiency anemia in subjects in need thereof (e.g., in patients with CKD).

Ferric citrate can be administered to a subject to improve one or more iron storage parameters (e.g., increase serum ferritin levels, increase transferrin saturation (TSAT), increase hemoglobin concentration) increase iron absorption, maintain iron stores, treat iron deficiency, treat anemia, reduce the need for IV iron and/or reduce the need for erythropoiesis-stimulating agents (ESAs).

In embodiments, the pharmaceutical composition disclosed herein can be administered to CKD patients. In embodiments, the pharmaceutical composition disclosed herein can be administered to CKD patients to improve one or more iron storage parameters, including to increase serum ferritin, to increase transferrin saturation (TSAT), and to increase hemoglobin concentration. In embodiments, the pharmaceutical composition disclosed herein can be administered to CKD patients to increase iron absorption. In embodiments, the pharmaceutical composition disclosed herein can be administered to CKD patients to maintain iron stores. In embodiments, the pharmaceutical composition disclosed herein can be administered to CKD patients to treat iron deficiency. In embodiments, the pharmaceutical composition disclosed herein can be administered to CKD patients to treat anemia. In embodiments, the pharmaceutical composition disclosed herein can be administered to CKD patients to reduce the need for IV iron and/or erythropoiesis-stimulating agents (ESAs).

In embodiments, provided herein are methods for treating iron deficiency anemia in a subject in need thereof, comprising administering to the subject an effective amount of ferric citrate. In embodiments, a subject is ≤ about 18 years of age. In embodiments, a subject has chronic kidney disease.

In certain embodiments, a subject treated for IDA in accordance with the methods described herein experiences a therapeutic benefit. In embodiments, a subject treated for IDA in accordance with the methods described herein experiences one, two, three or more, or all of the following effects: (i) an improvement in one or more symptoms of IDA; (ii) a reduction in the number of symptoms associated with IDA; (iii) a reduction in the duration of one or more symptoms; (iv) an improvement (e.g., an increase) in one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level; (v) a reduction in the administration of intravenous iron and/or an erythropoiesis stimulating agent; (vi) a decrease in iron deficiency; and/or (vii) a decrease or elimination of one, two, three, four or more symptoms of IDA. Symptoms of IDA include, but are not limited to, fatigue, dizziness, lightheadedness, pallor, hair loss, irritability, weakness, pica, brittle or grooved nails, dyspnea, anxiety, sadness, angina, constipation, sleepiness, tinnitus, mouth ulcers, Plummer-Vinson syndrome (painful atrophy of the mucous membrane covering the tongue, pharynx and esophagus), palpitations, hair loss, fainting or feeling faint, depression, twitching muscles, pale yellow skin, tingling (numbness) or burning sensations, missed menstrual cycle(s), heavy menstrual period(s), slow social development, glossitis, angular cheilitis, koilonychias, poor appetite, prurius, insomnia, dizziness, strange cravings for non-food items (e.g., dirt, ice, and clay), fast or irregular heartbeat, headaches, shortness of breath, cold hands and feet, impaired immune function, pagophagia, restless legs syndrome and combinations of the foregoing. In certain embodiments, a decrease in iron deficiency occurs as the total amount of iron in the body of the IDA patient is increased through the administration of the ferric citrate or a pharmaceutical composition thereof.

In embodiments, a subject is a child of ages 6 to <18 years. In embodiments, a subject has chronic kidney disease (CKD). In embodiments, a subject is on dialysis (e.g., chronic dialysis). In embodiments, a subject has non-dialysis-dependent CKD (ND-CKD) such as stage 3-5 CKD. In embodiments, a subject has dialysis-dependent CKD (DD-CKD). In embodiments, a subject has a hemoglobin (Hgb) of ≥8.5 and ≤11.5 g/dl (e.g., at screening and/or at commencement of therapy). In embodiments, a subject has a transferrin saturation (TSAT) ≤25% (e.g., at screening and/or at commencement of therapy). In embodiments, a subject has a ferritin level of ≤200 ng/ml (e.g., at screening and/or at commencement of therapy).

In embodiments, a subject of 6 to <13 years does not have a serum phosphorus level of ≤4.0 mg/dl (e.g., at screening and/or at commencement of therapy).

In embodiments, a subject of 13 to <18 years does not have a serum phosphorus level of ≤2.7 mg/dl (e.g., at screening and/or at commencement of therapy).

In embodiments, a subject receives ferric citrate as an oral dosage form (e.g., a tablet such as a coated tablet) comprising 1 g ferric citrate (210 mg of ferric iron). In embodiments, a subject receives ferric citrate as an oral dosage form (e.g., a tablet such as a coated tablet) comprising 250 mg ferric citrate (52.5 mg of ferric iron).

In embodiments, a method described herein results in a beneficial change from baseline in Hgb, TSAT, ferritin, serum phosphorus, calcium, and/or bicarbonate.

In embodiments, a method described herein results in a beneficial change from baseline in Hgb.

In embodiments, a method described herein results in a beneficial change from baseline in TSAT.

In embodiments, a method described herein results in a beneficial change from baseline in ferritin.

In embodiments, a method described herein results in a beneficial change from baseline in serum phosphorus.

In embodiments, a method described herein results in a beneficial change from baseline in calcium.

In embodiments, a method described herein results in a beneficial change from baseline in bicarbonate.

In embodiments, a method comprises administration of an initial (starting) dose to a subject. In embodiments, a method comprises weight-based dosing for a subject. In embodiments, a method comprises administration of ferric citrate according to any individual feature (e.g., any amount or modification) described in Tables 10 and 11 described herein, or any combination thereof.

In embodiments, a maximum daily dose is about three times the initial (starting) dose.

In embodiments, a subject of a weight that is about 12 kg to <40 kg receives an initial (starting) daily dose of about 750 mg ferric citrate. In embodiments, a dose may be modified by increments of 750 mg. In embodiments, a maximum daily dose is about 2250 mg. In embodiments, the ferric citrate is administered as 250 mg tablets (e.g., according to any formulation described herein).

In embodiments, a subject of a weight that is about 40 kg to <60 kg receives an initial (starting) daily dose of about 1500 mg ferric citrate. In embodiments, a dose may be modified by increments of 1500 mg. In embodiments, a maximum daily dose is about 4500 mg. In embodiments, the ferric citrate is administered as 250 mg tablets (e.g., according to any formulation described herein). In embodiments, the ferric citrate is administered as a combination of 250 mg tablets (e.g., according to any formulation described herein) and 1000 mg tablets (e.g., as described herein).

In embodiments, a subject of a weight that is about ≥60 kg receives an initial (starting) daily dose of about 3000 mg ferric citrate. In embodiments, a dose may be modified by increments of 3000 mg. In embodiments, a maximum daily dose is about 9000 mg. In embodiments, the ferric citrate is administered as 250 mg tablets (e.g., according to any formulation described herein). In embodiments, the ferric citrate is administered as 1000 mg tablets. In embodiments, the ferric citrate is administered as a combination of 250 mg tablets (e.g., according to any formulation described herein) and 1000 mg tablets (e.g., as described herein).

In embodiments, a daily dose of ferric citrate is modified. In embodiments, a daily dose of ferric citrate is modified based on various parameters (e.g., the iron and/or the serum phosphorus levels of the subject). In embodiments, a method comprises modification of the daily dose of ferric citrate according to any individual feature (e.g., any amount or modification) described in Table 12 or elsewhere in Example 4 as described herein, or any combination thereof.

In embodiments, a subject's daily dose of ferric citrate is not modified if the Hgb increase from baseline is ≥0.5 g/dl and/or Hgb level is ≥10 g/dl at a titration time point.

In embodiments, a subject's daily dose of ferric citrate will be modified (e.g., increased) based on the subject's increase in Hgb from baseline and/or Hgb level at a titration time point. In embodiments, a subject's daily dose of ferric citrate will be increased if the subject's increase in Hgb from baseline is <0.5 g/dl and Hgb level is <10 g/dl at a titration time point. In embodiments, a daily dose of ferric citrate will be increased. In embodiments, a subject's daily dose of ferric citrate will be increased if the subject's increase in Hgb from baseline is <0.5 g/dl and Hgb level is <10 g/dl at a titration time point. In embodiments, a daily dose of ferric citrate will be increased to a maximum daily dose. In embodiments, a subject having a body weight of about 12 kg to <40 kg has a dose increase of about 750 mg ferric citrate from an initial (starting) dose (e.g., an initial dose of about 750 mg). In embodiments, a subject having a body weight of about 12 kg to <40 kg has a second dose increase of about 750 mg ferric citrate or a total dose increase of about 1500 mg ferric citrate from an initial starting dose. In embodiments, a subject having a body weight of about 12 kg to <40 kg receives a maximum total daily dose of about 2250 mg ferric citrate. In embodiments, a subject having a body weight of about 40 kg to <60 kg has a dose increase of about 1500 mg ferric citrate from an initial (starting) dose (e.g., an initial dose of about 1500 mg). In embodiments, a subject having a body weight of about 40 kg to <60 kg has a second dose increase of about 1500 mg ferric citrate or a total dose increase of about 3000 mg ferric citrate from an initial starting dose. In embodiments, a subject having a body weight of about 40 kg to <60 kg receives a maximum total daily dose of about 4500 mg ferric citrate. In embodiments, a subject having a body weight of about ≥60 kg has a dose increase of about 3000 mg ferric citrate from an initial (starting) dose (e.g., an initial dose of about 3000 mg). In embodiments, a subject having a body weight of about ≥60 kg has a second dose increase of about 3000 mg ferric citrate or a total dose increase of about 6000 mg ferric citrate from an initial starting dose. In embodiments, a subject having a body weight of about ≥60 kg receives a maximum total daily dose of about 9000 mg ferric citrate.

In embodiments, a subject's daily dose of ferric citrate will be modified (e.g., increased) based on the subject's serum phosphorus level. In embodiments, a subject's daily dose of ferric citrate will be modified if the subject has a serum phosphorus level that is about 0.4 mg/dl above the lower limit of the age-related reference ranges (established in the KDOQI Clinical Practice Guideline for Nutrition in Children with CKD [NKF 2008]. In embodiments, a subject's daily dose of ferric citrate will be increased when serum phosphorus is >4.0 mg/dl in a subject of the age of 26 to <13 years. In embodiments, a subject's daily dose of ferric citrate will be increased when serum phosphorus is >2.7 mg/dl in a subject of the age of ≤13 to <18 years. In embodiments, a daily dose of ferric citrate will be increased to a maximum daily dose. In embodiments, a subject having a body weight of about 12 kg to <40 kg has a dose increase of about 750 mg ferric citrate from an initial (starting) dose (e.g., an initial dose of about 750 mg).

In embodiments, a subject having a body weight of about 12 kg to <40 kg has a second dose increase of about 750 mg ferric citrate or a total dose increase of about 1500 mg ferric citrate from an initial starting dose. In embodiments, a subject having a body weight of about 12 kg to <40 kg receives a maximum total daily dose of about 2250 mg ferric citrate. In embodiments, a subject having a body weight of about 40 kg to <60 kg has a dose increase of about 1500 mg ferric citrate from an initial (starting) dose (e.g., an initial dose of about 1500 mg). In embodiments, a subject having a body weight of about 40 kg to <60 kg has a second dose increase of about 1500 mg ferric citrate or a total dose increase of about 3000 mg ferric citrate from an initial starting dose. In embodiments, a subject having a body weight of about 40 kg to <60 kg receives a maximum total daily dose of about 4500 mg ferric citrate. In embodiments, a subject having a body weight of about ≥60 kg has a dose increase of about 3000 mg ferric citrate from an initial (starting) dose (e.g., an initial dose of about 3000 mg). In embodiments, a subject having a body weight of about ≥60 kg has a second dose increase of about 3000 mg ferric citrate or a total dose increase of about 6000 mg ferric citrate from an initial starting dose. In embodiments, a subject having a body weight of about ≥60 kg receives a maximum total daily dose of about 9000 mg ferric citrate.

In embodiments treatment with ferric citrate is discontinued or paused based on serum phosphorus levels. In embodiments, administration of ferric citrate is paused or discontinued for a subject having an age of ≥6 to <13 years if the serum phosphorus is not >4.0 mg/dl. In embodiments, administration of ferric citrate is paused or discontinued for a subject having an age of ≥13 to <18 years if the serum phosphorus is not >2.3 mg/dl.

In embodiments, treatment with ferric citrate is discontinued or paused based on TSAT. In embodiments, a daily dose of ferric citrate is modified based on TSAT. In embodiments, a treatment modification occurs if a subject has a TSAT is ≥50% and <70% at an initial laboratory test and a follow-up laboratory test and the results are confirmed at a subsequent follow-up laboratory test. In embodiments, administration of ferric citrate is discontinued for a subject having a repeat TSAT that is ≥70%. In embodiments, a daily dose of ferric citrate is modified (e.g., reduced). for a subject having a repeat TSAT that is ≥50 and <70%.

In embodiments, treatment with ferric citrate is not modified based on serum ferritin (e.g., elevated serum ferritin) in the absence of undesirable Hgb or TSAT values (e.g., as described herein).

Doses and Dosing Regimens

Suitable dosing regimens include those described herein, including but not limited to the exemplary dosing regimens provided for treatment of hyperphosphatemia and/or iron deficiency anemia described herein.

In embodiments, a subject receives a dose of about 1 g, about 2 g, about 3 g, about 4 g, about 5 g, about 6 g, about 7 g, about 8 g, about 9 g, about 10 g, about 11 g, about 12 g, about 13 g, about 14 g, or about 15 g of ferric citrate. In embodiments, a subject receives a dose of no more than about 1 g, about 2 g, about 3 g, about 4 g, about 5 g, about 6 g, about 7 g, about 8 g, about 9 g, about 10 g, about 11 g, about 12 g, about 13 g, about 14 g, or about 15 g of ferric citrate. In embodiments, a subject receives a dose of at least about 1 g, about 2 g, about 3 g, about 4 g, about 5 g, about 6 g, about 7 g, about 8 g, about 9 g, about 10 g, about 11 g, about 12 g, about 13 g, about 14 g, or about 15 g of ferric citrate. In embodiments, a subject receives a dose of at least about 1 g to about 3 g, about 3 g to about 5 g, about 5 g to about 8 g, about 8 g to about 12 g, or about 12 g to about 15 g of ferric citrate. In embodiments, a subject receives a dose of no more than about 1 g to about 3 g, about 3 g to about 5 g, about 5 g to about 8 g, about 8 g to about 12 g, or about 12 g to about 15 g of ferric citrate.

In embodiments, ferric citrate or a pharmaceutical composition (e.g., a tablet) thereof is administered continuously and/or indefinitely.

In certain embodiments, ferric citrate or a pharmaceutical composition (e.g., a tablet) thereof is administered 3 times per day. In certain embodiments, ferric citrate or a pharmaceutical composition (e.g., a tablet) thereof is administered once daily. In certain embodiments, ferric citrate or a pharmaceutical composition (e.g., a tablet) thereof is administered three times per week. In certain alternative embodiments, ferric citrate or a pharmaceutical composition (e.g., a tablet) thereof is administered on an as needed basis.

In certain embodiments, ferric citrate or a pharmaceutical composition (e.g., a tablet) thereof is administered for a period of time, such as 1 month, 2 months, 3 months, 4 months, 5 months, 6 months, 9 months, 12 months or more.

Combination Therapy with a HIF-PH Inhibitor

In embodiments, a subject receiving a ferric citrate may also be administered a compound that is a hypoxia-inducible factor-prolyl hydroxylase (HIF-PH) inhibitor.

In embodiments, a subject is receiving an iron-containing composition prior to commencement of therapy with a HIF-PH inhibitor. In embodiments, a subject receives an iron-containing composition after commencing therapy with a HIF-PH inhibitor.

In embodiments, a patient's treatment with a HIF-PH inhibitor is initiated at the same time as said patient's treatment with an iron-containing composition.

In embodiments, a subject is administered an iron-containing composition for treating a disease or condition in the patient that was present at the time treatment with a HIF-PH inhibitor was commenced. In embodiments, a subject is administered an iron-containing composition for treating or preventing a disease or condition in the patient that was not present at the time treatment with a HIF-PH inhibitor was commenced (e.g., the disease or condition developed after treatment with the first compound was commenced). In embodiments, a subject is administered an iron-containing composition for treating or preventing a disease or condition in the patient induced by treatment with a HIF-PH inhibitor. In embodiments, a subject is administered an iron-containing composition for treating or preventing a disease or condition in the patient that arises independently of treatment with a HIF-PH inhibitor.

In embodiments, a subject is administered a HIF-PH inhibitor for treating a disease or condition in the patient that was present at the time treatment with an iron-containing composition was commenced. In embodiments, a subject is administered a HIF-PH inhibitor for treating or preventing a disease or condition in the patient that was not present at the time treatment with an iron-containing composition was commenced (e.g., the disease or condition developed after treatment with the first compound was commenced). In embodiments, a subject is administered a HIF-PH inhibitor for treating or preventing a disease or condition in the patient induced by treatment with an iron-containing composition. In embodiments, a subject is administered a HIF-PH inhibitor for treating or preventing a disease or condition in the patient that arises independently of treatment with an iron-containing composition.

In embodiments, an iron-containing composition is administered before (e.g., at least about four hours after) the administration of a HIF-PH inhibitor. In embodiments, an iron-containing composition is administered after (e.g., at least about four hours after) the administration of a HIF-PH inhibitor.

Exemplary methods include those described in PCT/US22/11668, which is hereby incorporated by reference in its entirety.

Exemplary HIF-PH Inhibitor Compounds

Non-limiting examples of HIF-PH inhibitors include but are not limited to vadadustat (AKB-6548), roxadustat (FG-4592), daprodustat (GSK-12788363), molidustat (BAY 85-3934), enarodustat (JTZ-951), and desidustat (ZYAN1), or a pharmaceutically acceptable salt thereof, and compounds described in U.S. Pat. Nos. 8,759,345, 8,937,078, 8,796,263, 9,273,034, 8,530,404, 7,696,223, 7,629,357, 8,927,591, 8,269,008, 8,952,160, 8,952,160, 8,927,591, 8,921,389, 8,916,585, 8,703,795, 8,921,389, 7,662,854, and 9,040,522; in International Publication No. WO2020/072645; in U.S. Provisional Patent Application Nos. 63/125,661, 63/125,642, 62/992,585, 62/992,606, 62/992,616, 63/081,005, and 63/065,642; and in “Recent Advances in Developing Inhibitors for Hypoxia-Inducible Factor Prolyl Hydroxylases and Their Therapeutic Implications” (Kim et al. Molecules 2015, 20, 20551-20568; see, e.g., any of the compounds described therein, including any of the compounds described in any of Tables 2, 3, or 4), each of which is hereby incorporated by reference in its entirety. In embodiments, a suitable compound is described in any of International Publication No. WO2020/072645; and in U.S. Provisional Patent Application Nos. 63/125,661, 63/125,642, 62/992,585, 62/992,606, 62/992,616, 63/081,005, and 63/065,642.

### Vadadustat and Related Compounds

Exemplary HIF-PH inhibitor compounds that can be used in any of the methods described herein include those described in U.S. Pat. Nos. 7,811,595, 8,343,952, 8,323,671, 8,598,210, 8,722,895, 8,940,773, and 9,598,370; and in U.S. Publication No. US 20190192494A1, each of which is incorporated by reference in its entirety. In embodiments, a compound, or pharmaceutically acceptable salt thereof, is described in any of claims 1-32 of U.S. Pat. No. 7,811,595.

In embodiments, a HIF-PH inhibitor is {[5-(3-chlorophenyl)-3-hydroxypyridine-2-carbonyl]amino}acetic acid (Compound 1), or a pharmaceutically acceptable salt thereof. Compound 1, also referred to as vadadustat or AKB-6548, has the following structure:

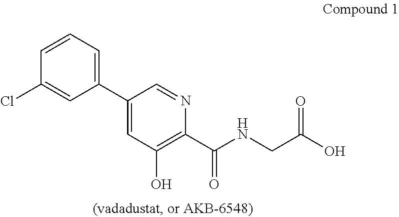 [SMILES: O=C(O)CNC(=O)c1ncc(-c2cccc(Cl)c2)cc1O]

### Roxadustat and Related Compounds

Still other HIF-PH inhibitor compounds that can be useful in the methods described herein include those described in U.S. Pat. No. 7,323,475, which is incorporated by reference in its entirety. In embodiments, a compound, or a pharmaceutically acceptable salt thereof, is described in any of claims 1-46 of U.S. Pat. No. 7,323,475.

In embodiments, a HIF-PH inhibitor is (1-methyl-4-hydroxy-7-phenoxy-isoquinoline-3-carbonyl)-amino-acetic acid (Compound 2), or a pharmaceutically acceptable salt thereof. Compound 2, also referred to as roxadustat, or FG-4592, has the following structure:

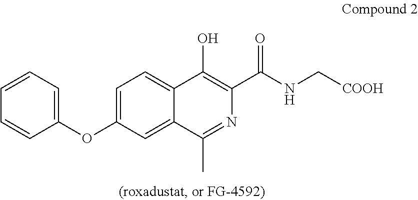 [SMILES: Cc1nc(C(=O)NCC(=O)O)c(O)c2ccc(Oc3ccccc3)cc12]

### Daprodustat and Related Compounds

Still other HIF-PH inhibitor compounds that can be useful in the methods described herein include those described in U.S. Pat. No. 8,324,208, which is incorporated by reference in its entirety. In embodiments, a compound, or a pharmaceutically acceptable salt thereof, is described in any of claims 1-16 of U.S. Pat. No. 8,324,208.

In embodiments, a HIF-PH inhibitor is N-(1,3-Dicyclohexyl-6-hydroxy-2,4-dioxo-1,2,3,4-tetrahydro-5-pyrimidinyl)carbonylglycine (Compound 3), or a pharmaceutically acceptable salt thereof. Compound 3, also referred to as daprodustat, or GSK-12788363, has the following structure:

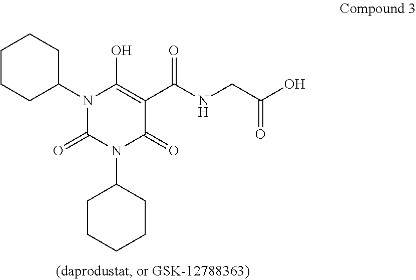 [SMILES: O=C(O)CNC(=O)c1c(O)n(C2CCCCC2)c(=O)n(C2CCCCC2)c1=O]

### Molidustat and Related Compounds

Still other HIF-PH inhibitor compounds that can be useful in the methods described herein include those described in U.S. Pat. No. 8,389,520, which is incorporated by reference in its entirety. In embodiments, a compound, or a pharmaceutically acceptable salt thereof, is described in any of claims 1-10 of U.S. Pat. No. 8,389,520.

In embodiments, a HIF-PH inhibitor is 2-(6-Morpholin-4-ylpyrimidin-4-yl)-4-(1H-1,2,3-triazol-1-yl)-1,2-dihydro-3H-pyrazol-3-one (Compound 4), or a pharmaceutically acceptable salt thereof. Compound 4, also referred to as molidustat, or BAY 85-3934, has the following structure:

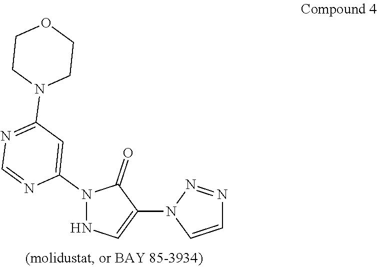 [SMILES: O=c1c(-n2ccnn2)c[nH]n1-c1cc(N2CCOCC2)ncn1]

### Enarodustat and Related Compounds

Exemplary HIF-PH compounds that can be used in any of the methods described herein include those described in U.S. Pat. No. 8,283,465, U.S. Publication No. US20160145254A1, and U.S. Publication No. US20200017492A1, each of which is incorporated by reference in its entirety. In embodiments, a compound, or pharmaceutically acceptable salt thereof, is described in any of claims 1-30 of U.S. Pat. No. 8,283,465.

In embodiments, a HIF-PH inhibitor is [(7-hydroxy-5-phenethyl [1,2,4]triazolo [1,5-a]pyridine-8-carbonyl)aminoacetic acid (Compound 5), or a pharmaceutically acceptable salt thereof. Compound 5, also referred to as enarodustat, or JTZ-951, has the following structure:

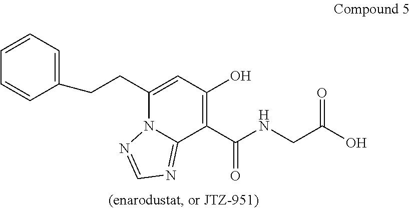 [SMILES: O=C(O)CNC(=O)c1c(O)cc(CCc2ccccc2)n2ncnc12]

### Desidustat and Related Compounds

Exemplary HIF-PH inhibitor compounds that can be used in any of the methods described herein include those described in U.S. Pat. No. 9,394,300, and U.S. Publication No. US 20190359574A1, each of which is incorporated by reference in its entirety. In embodiments, a compound, or pharmaceutically acceptable salt thereof, is described in any of claims 1-10 of U.S. Pat. No. 9,394,300.

In embodiments, a HIF-PH inhibitor is 2-(1-(cyclopropylmethoxy)-4-hydroxy-2-oxo-1,2-dihydroquinoline-3-carboxamido) acetic acid (Compound 6), or a pharmaceutically acceptable salt thereof. Compound 6, also referred to as enarodustat, or ZYAN1, has the following structure:

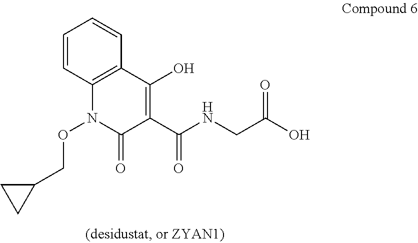 [SMILES: O=C(O)CNC(=O)c1c(O)c2ccccc2n(OCC2CC2)c1=O]

HIF-PH inhibitor compounds described herein can be used with the ferric citrate compositions and formulations provided herein.

Doses and Dosing Regimens of a HIF-PH inhibitor

The specific doses for uses of a HIF-PH inhibitor as described herein can be administered in any manner known to the skilled artisan. Exemplary doses are provided herein, including in the Examples.

In embodiments, a disease or condition as described herein, may be treated by administering a HIF-PH inhibitor (e.g., any described herein, including any one of Compounds 1-6 such as Compound 1) to a patient in need thereof. In embodiments, the dose of a HIF-PH inhibitor is about 1 mg to about 1500 mg. In embodiments, the dose of a HIF-PH inhibitor is about 1 mg to about 1800 mg. In embodiments, the dose of a HIF-PH inhibitor is about 1 mg to about 10 mg, about 10 mg to about 20 mg, about 20 mg to about 50 mg, about 50 mg to about 100 mg, about 100 mg to about 200 mg, about 200 mg to about 300 mg, about 300 mg to about 400 mg, about 400 mg to about 600 mg, about 60 mg to about 800 mg, about 800 mg to about 1000 mg, about 1000 mg to about 1200 mg, about 1200 mg to about 1500 mg, about 1500 mg to about 1800 mg.

In embodiments, the dose of a HIF-PH inhibitor (e.g., any described herein, including any one of Compounds 1-6 such as Compound 1) is about 150 mg to about 600 mg, about 150 mg to about 750 mg, about 150 mg to about 900 mg, about 150 mg to about 1200 mg, about 150 mg to about 1500 mg, about 75 mg to about 1200 mg, about 75 mg to about 1500 mg, or about 75 mg to about 1800 mg. In embodiments, the dose of a HIF-PH inhibitor is at least about 150 mg to about 600 mg, about 150 mg to about 750 mg, about 150 mg to about 900 mg, about 150 mg to about 1200 mg, about 150 mg to about 1500 mg, about 75 mg to about 1200 mg, about 75 mg to about 1500 mg, or about 75 mg to about 1800 mg. In embodiments, the dose of a HIF-PH inhibitor is no more than about 150 mg to about 600 mg, about 150 mg to about 750 mg, about 150 mg to about 900 mg, about 150 mg to about 1200 mg, about 150 mg to about 1500 mg, about 75 mg to about 1200 mg, about 75 mg to about 1500 mg, or about 75 mg to about 1800 mg.

In embodiments, HIF-PH inhibitors include (but are not limited to) compounds such as vadadustat (AKB-6548), roxadustat (FG-4592), daprodustat (GSK-12788363), molidustat (BAY 85-3934), enarodustat (JTZ-951), and desidustat (ZYAN1), or a pharmaceutically acceptable salt thereof. In embodiments, a HIF-PH inhibitor is daprodustat, roxadustat, or vadadustat. In embodiments, a HIF-PH inhibitor is vadadustat.

In embodiments, a HIF-PH inhibitor is vadadustat. In embodiments, the dose of vadadustat is about 150 mg to about 600 mg, about 150 mg to 750 mg, about 150 mg to 900 mg, about 150 mg to 1200 mg, about 150 mg to 1500 mg, about 75 mg to 1200 mg, about 75 mg to 1500 mg, or about 75 mg to 1800 mg. In embodiments, the dose of vadadustat is about 75 mg to about 1200 mg, about 150 mg to about 600 mg, or about 150 mg to about 750 mg. In embodiments, the dose of vadadustat is about 75 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, or about 1800 mg. In embodiments, the dose of vadadustat is at least about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, or about 1800 mg. In embodiments, the dose of vadadustat is no more than about 75 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, or about 1800 mg.

Timing of Administration

In embodiments of methods described herein, combination therapy may comprise administration of a compound (e.g., a first compound) that is a HIF-PH inhibitor (e.g., vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof) and a compound (e.g., a second compound) that is ferric citrate.

In embodiments, administration of a compound (e.g., a first compound) and the other compound (e.g., a second compound) occurs concomitantly (concomitant administration). In embodiments, concomitant administration of a compound (e.g., a first compound) and administration of the other compound (e.g., a second compound) occur within a time period that is no more than one about hour (e.g., no more than about 1, 5, 10, 15, 20, 25 or 30 minutes). In embodiments, concomitant administration of a compound (e.g., a first compound) and the other compound (e.g., a second compound) occurs simultaneously (simultaneous administration) where both compounds are administered at the same time. In embodiments, the combination therapy is administered for at least 7, 14, 21, or 28 days to a patient in need thereof.

In embodiments, administration of a compound (e.g., a first compound) and the other compound (e.g., a second compound) occurs sequentially (sequential administration). In embodiments, a first compound is given at least about 1, 2, or 4 hours before and/or after taking a second compound. In embodiments, a first compound is given at least about 4 hours before and/or after taking a second compound. In embodiments, the combination therapy is administered for at least 7, 14, 21, or 28 days to a patient in need thereof.

In embodiments, a first compound is a HIF-PH inhibitor that is vadadustat. In embodiments, vadadustat is administered at a total daily dose of about 150-600 mg. In embodiments, a second compound is ferric citrate that is administered at a total daily dose of such as those described herein. In embodiments, the combination therapy is administered for at least 7, 14, 21, or 28 days to a patient in need thereof. In embodiments, an improved (e.g., synergistic) therapeutic effect can be observed about 5, 10, 15, or 20 days after commencement of the treatment. In embodiments, an improved (e.g., synergistic) therapeutic effect can be observed for about 5, 10, 15, 20, or more than 20 days during the treatment.

In embodiments, a patient has iron deficiency anemia (e.g., a patient may have iron deficiency anemia at commencement of treatment according to methods described herein). In embodiments, a patient has anemia secondary to or associated with chronic kidney disease (renal anemia). In embodiments, a patient has non-dialysis dependent chronic kidney disease (NDD-CKD). In embodiments, a patient has dialysis dependent chronic kidney disease (DD-CKD).

### Exemplification

The following examples describe the preparation and properties of the ferric citrate tablets described herein. It will be apparent to those skilled in the art that many modifications, both to materials and methods, can be practiced without departing from the scope of the disclosure.

### Example 1—Ferric Citrate 250 mg Pediatric Tablet

Provided herein are exemplary formulations according to any one of the embodiments described herein. Each of Tables 1A and 1B summarizes the formulation of an exemplary ferric citrate 250 mg pediatric tablet.

TABLE 1A
Ferric Citrate 250 mg Pediatric Tablets (Formulation 1)

| | | | |
|---|---|---|---|
| | Component | Amount per 250 | Content |
| Component | Function | mg dose (mg) | (% w/w) |

Intragranular Blend

| | | | |
|---|---|---|---|
| Ferric Citrate | Active | 250.0 | 71.4 |
| Copovidone (binder) | Binder | 17.9 | 5.1 |
| Microcrystalline Cellulose | Filler | 71.6 | 20.5 |
| Crospovidone | Disintegrant | 7.1 | 2.0 |
| Magnesium Stearate | Lubricant | 0.9 | 0.3 |

Extragranular Blend

| | | | |
|---|---|---|---|
| Colloidal Silicon Dioxide | Glidant | 0.7 | 0.2 |
| Magnesium Stearate | Lubricant | 1.8 | 0.5 |

| | | |
|---|---|---|
| Total Core Tablet Weight | 350.0 | 100.0 |

Film Coat

| | | | |
|---|---|---|---|
| Ferric Citrate Core Tablet | N/A | 350.0 | 96.2 |
| Opadry ® QX Pink | Tablet Coating | 14.0 | 3.8 |

| | | |
|---|---|---|
| Total Coated Tablet Weight | 364.0 | 100.0 |

TABLE 1B
Ferric Citrate 250 mg Pediatric Tablets (Formulation 2)

| | | | |
|---|---|---|---|
| | Component | Amount per 250 | Content |
| Component | Function | mg dose (mg) | (% w/w) |

Intragranular Blend

| | | | |
|---|---|---|---|
| Ferric Citrate | Active | 250.0 | 71.4 |
| Copovidone (binder) | Binder | 17.9 | 5.1 |
| Microcrystalline Cellulose | Filler | 71.6 | 20.5 |
| Crospovidone | Disintegrant | 7.1 | 2.0 |
| Magnesium Stearate | Lubricant | 0.9 | 0.3 |

Extragranular Blend

| | | | |
|---|---|---|---|
| Colloidal Silicon Dioxide | Glidant | 0.7 | 0.2 |
| Magnesium Stearate | Lubricant | 1.8 | 0.5 |

| | | |
|---|---|---|
| Total Core Tablet Weight | 350.0 | 100.0 |

Film Coat

| | | | |
|---|---|---|---|
| Ferric Citrate Core Tablet | N/A | 350.0 | 96.2 |
| Opadry ® Purple | Tablet Coating | 14.0 | 3.8 |

| | | |
|---|---|---|
| Total Coated Tablet Weight | 364.0 | 100.0 |

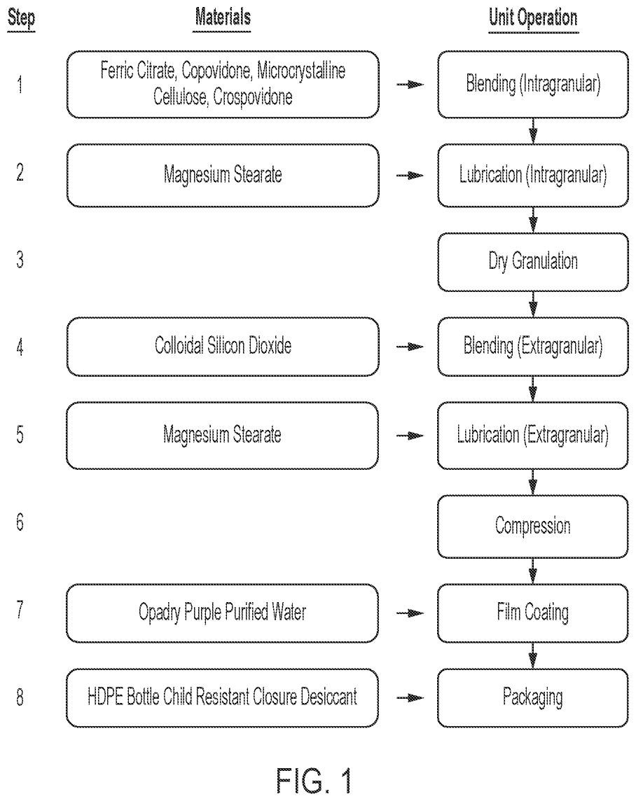

An exemplary method for preparing tablets comprising ferric citrate (e.g., tablets according to Formulation 2) is described in FIG. 1. In embodiments, tablets were prepared according to the following steps:
1. Blending (Intragranular)
   Sieve ferric citrate, copovidone, microcrystalline cellulose, and crospovidone and blend them in a tumble blender.
2. Lubrication (Intragranular)
   Sieve magnesium stearate and blend with components from Step 1 to make an intragranular blend.
3. Dry Granulation
   Pass the intragranular blend through a roller compactor to form ribbons and pass through the in-built mill to form granules.
4. Blending (Extragranular)
   Sieve colloidal silicon dioxide and blend with granules from step 3 in a tumble blender.
5. Lubrication (Extragranular)
   Sieve magnesium stearate and blend with components from Step 4 to make a final blend.
6. Compression
   The lubricated blend from Step 5 is used to compress ferric citrate 250 mg tablets using a rotary tablet press.
7. Coating
   The core tablets from Step 6 are placed into a coating pan, and an aqueous solution of Opadry® Purple in purified water is prepared. The coating solution is sprayed onto the core tablets and then dried.

In embodiments, tablets comprising ferric citrate (e.g., according to Formulation 1) were prepared according to below steps:
Pre-blending:
   Ferric citrate, crospovidone, copovidone were screened prior to blending.
   The screened material was blended in 3 cu.ft V-Blender for 20 minutes.
   Magnesium stearate (intra granular) was passed through a #30 mesh screen and blended with the above material for 3 minutes.
   The above material was charged into 2 cu.ft V-blender.
Blending:
   colloidal silicon dioxide was passed through a #30 mesh screen and added to the above material in 2 cu.ft V-blender.
   The above material was blended for 8 minutes.
   Magnesium stearate was passed through a #30 mesh screen and blended for 2 minutes and 30 seconds with the above material in 2 cu.ft V-blender.
Compression:
   The material was compressed with Korsch XL-100 press using modified oval tooling to form tablets.
Coating:
   The tablets were coated with O'Hara Labcoat 24″ coating pan (3.0% coating weight gain).

Table 2 summarizes certain properties of the tablets.

TABLE 2
Exemplary Tablet Formulation

| Tablet Properties | |
|---|---|
| Individual tablet weight (mg) | 343.2-357-9 |
| Individual tablet thickness (mm) | 4.63-4.79 |
| Individual tablet hardness (Kp) | 13.4-17.2 |
| Friability | 0.07% |
| Disintegration Time (min) | 11-12 min |
| Release testing | |
| Water content | 13.6% |
| Citrate content | 39.8% |
| Dissolution (Q = 80% in less than 60 minutes) | 96% |

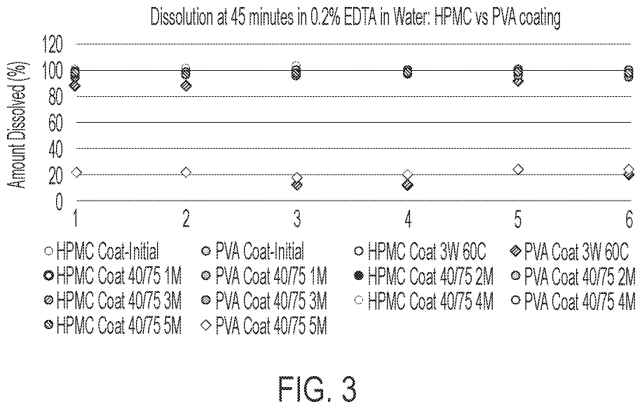

Table 3 provides exemplary dissolution data for tablet coatings comprising different polymeric binding agents, where Tablet Formulation 03K140049 comprises a HPMC based coat and Tablet Formulation 85F140215 comprises a PVA based coat, but otherwise comprise the same formulation of core tablet as Formulation 2 described herein. The two formulations were used for an accelerated stability study (40° C./75% RH for 6 months or 60° C. for 3 weeks) to compare the dissolution performance. Likewise, FIG. 3 illustrates the dissolution results of film-coated Ferric Citrate 250 mg Tablets having a HPMC- or PVA-based coating material under accelerated storage conditions (n=6). Exemplary HPMC-based coatings include Opadry® Purple.

TABLE 3
Dissolution Results of Film-coated Ferric Citrate 250 mg Tablets Between
HPMC and PVA Based Coating Material Under Accelerated Storage Conditions

| | | Amount Dissolved (%) after 45 minutes, in 0.2% EDTA in Water | | | | | |
|---|---|---|---|---|---|---|---|
| | Storage | (Q = 80% in 45 minutes, n = 6) | | | | | |
| Lot # | Condition | T0 | | | 3W | | |
| 03K140049 | 60° C. | 99, 101, 102, 101, 100, 100 | | | 98, 99, 100, 100, 100, 100 | | |
| HPMC | | | | | | | |
| based | | | | | | | |
| 85F140215 | 60° C. | 99, 98, 100, 99, 101, 99 | | | 90, 89, 11, 12, 92, 20 | | |
| PVA based | | | | | | | |
| | | Amount Dissolved (%) after 45 minutes, in 0.2% EDTA in Water | | | | | |
| | Storage | (Q = 80% in 45 minutes, n = 6) | | | | | |
| Lot # | Condition | T0 | 1M | 2M | 3M | 4M | 5M |
| 03K140049 | 40° C./ | 99, 101, | 98, 100, | 98, 98, | 100, | 101, 102, | 99, 97, |
| HPMC | 75% RH | 102, 101, | 99, 99, | 97, 99, | 101, 99, | 104, 100, | 98, 99, |
| based | | 100, 100 | 100, 100 | 99, 97 | 98, 97, | 99, 101 | 100, 99 |
| | | | | | 98 | | |
| 85F140215 | 40° C./ | 99, 98, | 101, | 95, 100, | 97, 98, | 100, 101, | 21, 22, |
| PVA based | 75% RH | 100, 99, | 100, 99, | 101, 98, | 96, 98, | 100, 101, | 18, 21, |
| | | 101, 99 | 99, 99, | 98, 100 | 96, 95 | 101, 101 | 23, 23 |
| | | | 101 | | | | |

### Example 2—Coated Granule Formulations Comprising Ferric Citrate

The same composition of granules can be used for granule and tablet with dry granulation (roller compaction and slugging) as a main processing step.

The granules were coated via fluid bed coating process inside the Wurster column (bottom spray coating) with OPADRY® QX Purple coating system to evaluate granule robustness and to mask the taste and color of API.

A top spray wet granulation was attempted using only API with aqueous binder solution to improve the granules strength. The addition of binder (5% wt/wt) improved flow of the granules and resulted in non-uniform color of the granules with some granules showing dark color. These granules were taken for fluid bed coating (top spray and Wurster column) with 5% and 10% wt/wt gain. With bottom (Wurster) coating and 10% weight gain, uniform color and particle size distribution with least amount of fines were observed.

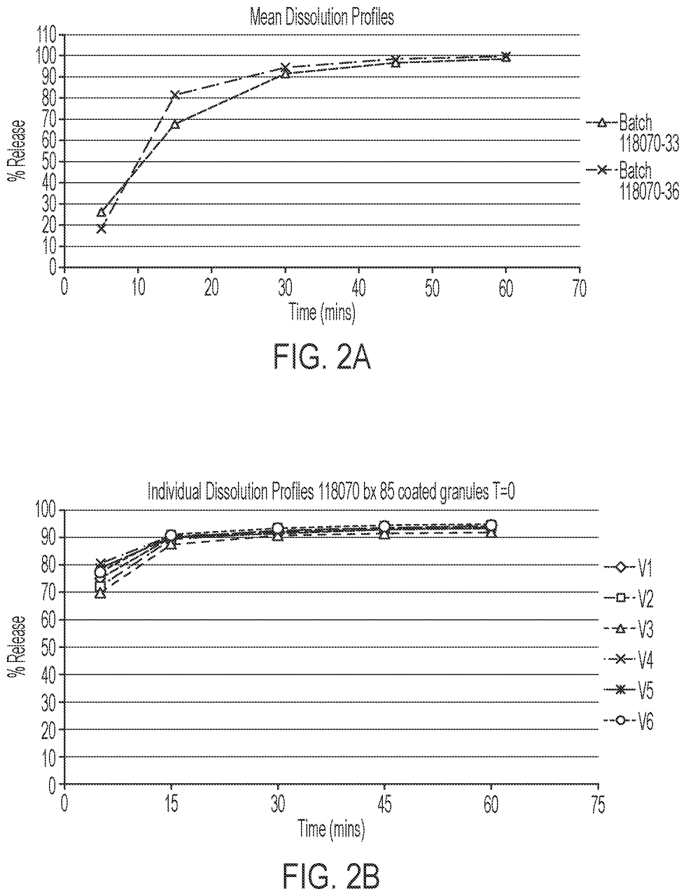

Granules were coated (Auryxia granules, batch #118070-33 with top spray process and Quotient granules, batch #118070-36 with bottom spray coating) using with OPADRY® QX Purple coating system to 10% weight gain. FIG. 2A shows the dissolution profile of these coated granules.

Quotient coated granules (Batch #118070-85) was coated with new formula of OPADRY® QX Purple. The resulting granules passed the required specification for assay, related substances and content uniformity. The passing dissolution result is presented in FIG. 2B.

The composition of granules used for Wurster coating are summarized in Table 4.

TABLE 4
Composition of Granules

| Component | Function | % w/w |
|---|---|---|
| QFW01 API | Active Pharmaceutical | 72.3 |
| | Ingredient | |
| Coprovidone VA 64 | Binder | 5.2 |
| Microcrystalline Cellulose PH 105 | Filler | 21.5 |
| Magnesium Stearate (Powder) Kosher | Lubricant | 1.0 |
| Passover Hyqual 5712, NF/Ph.Eur./JP | | |

The coated granules disintegrated in all foods examined (green beans, apple sauce, cream of wheat, angel delight, carrot and potato). After 5 minutes some of the granules began to darken slightly and by 10 minutes the observed leaching was significantly more pronounced. The stability of the coated granules passed specifications at T=1 month but due to failing compatibility in food, the study was terminated.

An updated granule composition is summarized in Table 5. As shown in Table 5, disintegrant was added and lubricant level decreased from 1% to 0.3%. The coating system chosen to proceed with was EPO. The repeated testing of dissolution of granules have shown passing specification for 5% coated granules and borderline dissolution for the 10% coated granules. However, food examination for coated granules with EPO coating system showed leaching at 5-minute time point in all foods examined (green peas, apple sauce, rice cereal). More noticeable change in color was seen with granules sprinkled on food compared to the granules mixed in with food.

TABLE 5
Core granules composition

| | Theoretical Composition |
|---|---|
| Component | (% w/w) |
| Ferric Citrate | 71.9 |
| Copovidone, NF/EP (Kollidon VA 64) | 5.2 |
| Microcrystalline Cellulose, NF/Ph. | 20.6 |
| Eur. (Avicel PH-105) | |
| Crospovidone, NF (Polyplasdone XL) | 2.0 |
| Magnesium Stearate (Powder) Kosher | 0.3 |
| Passover Hyqual 5712, NF/Ph.Eur./JP | |
| Or Magnesium Stearate, NF/Ph.Eur. | |
| (Kosher Passover Hyqual, 5712) | |
| Total | 100 |

### Example 3—Ferric Citrate in Children with Hyperphosphatemia Related to Chronic Kidney Disease [KRX-0502-308]

This is a phase 3, multicenter, 36-week, single-group, open-label study of ferric citrate in children (ages 6 to <18 years) with hyperphosphatemia related to CKD, with the majority (at least 60%) on chronic dialysis.

The study consists of a Screening period of up to 6 weeks, which will include a Washout period for otherwise eligible subjects who are on a phosphate binder, a 36-week Treatment period and a 30 day safety follow-up period after the last dose of study drug. After Screening, study visits will occur every week for the first month, and every 2 weeks thereafter. All subjects will receive ferric citrate; starting doses will be based on body weight categories, and doses will be adjusted per dose titration guidelines to age-appropriate serum phosphorus targets.

Adverse events (AEs) will be monitored including gastrointestinal AEs of special interest. Laboratory assessments will be conducted to monitor serum phosphorus, calcium, parathyroid hormone, iron parameters, liver tests, and other clinically relevant standard laboratory measures.

Ferric citrate doses may be reduced or discontinued for individual subjects, if necessary, on the basis of AEs or abnormal laboratory results. Concomitant use of intravenous (IV) iron is permitted if necessary; guidelines for IV iron use are provided. Oral iron other than study drug will not be permitted during the study. Multivitamins containing iron are permitted.

Main Criteria for Inclusion:
1. Age 6 years to <18 years at Screening.
2. Weight≥12 kg at Screening.
3. CKD requiring dialysis, or CKD not on dialysis with an estimated glomerular filtration rate (eGFR)<30 ml/min/1.73 m2 at Screening.
4. a) If subject is on phosphate binder(s) at Screening:
   At Visit 1, the subject, if eligible per all other inclusion and exclusion criteria, will discontinue phosphate binder(s) and proceed to the Washout period.
   After at least 1 week of washout (ie, at Visit 1a or Visit 1b), serum phosphorus must be:
      −6 to <13 years: >5.8 mg/dl.
      −13 to <18 years: >4.5 mg/dl.
b) If subject is not on a phosphate binder at Screening:
   At Visit 1, serum phosphorus must be:
      6 to <13 years: >5.8 mg/dl.
      −13 to <18 years: >4.5 mg/dl.
5. Transferrin saturation (TSAT)<50% at Screening.
6. Serum ferritin <500 ng/ml at Screening

Ferric citrate will be supplied as:
Tablets containing 1 g ferric citrate (210 mg of ferric iron).
Tablets containing 250 mg ferric citrate (52.5 mg ferric iron).

Primary Endpoints

Safety and tolerability evaluations will be based on:
Incidence, seriousness, and intensity of treatment-emergent AEs, including GI AEs of special interest.
Clinically significant laboratory abnormalities or changes in laboratory results during the Treatment period.
Treatment-emergent AEs leading to discontinuation of ferric citrate.

Secondary Endpoints
Change from baseline in serum phosphorus to Week 36/early termination (ET).

To account for significant differences in body weight across the broad range of pediatric subjects to be enrolled, dosing will be based on body weight categories, as detailed in Tables 6 and 7. The starting dose of ferric citrate ranges from one 6th of the adult starting dose (in the lowest body weight category) to the full adult starting dose (in the highest weight category). The approximate weight-based equivalents of each starting dose range from 38 to 100 mg/kg/day. The maximum dose of ferric citrate allowed in this study is 3 times the starting dose for all but the highest body weight category. For the highest body weight category, the maximum dose is twice the starting dose, which is consistent with the approved dosing for adults.

TABLE 6

| | Ferric Citrate Dosage (mg/day) | | |
|---|---|---|---|
| Body Weight at | | Titration | Maximum |
| Screening (kg) | Starting dose | Increments | Dose |
| 12 to <20 | 1000 | ±250, +1000 | 2500 |
| 20 to <40 | 2000 | ±500, +2000 | 5000 |
| 40 to <60 | 3000 | ±1000, +3000 | 9000 |
| ≥60 | 6000 | ±1000, +6000 | 12000 |

TABLE 7

| Body Weight at | Starting dose: | Total Daily Starting |
|---|---|---|
| Screening (kg) | Total Daily Dose (mg) | Dose, by Formulation |
| 12 to <20 | 1000 | 250-mg tablets: 4 tablets |
| 20 to <40 | 2000 | 250-mg tablets: 8 tablets |
| 40 to <60 | 3000 | 1-g tablets: 3 tablets |
| ≥60 | 6000 | 1-g tablets: 6 tablets |

Subjects will take ferric citrate orally with food (meals or snacks) or within 1 hour after eating. Tablets must be swallowed whole, without splitting, crushing, or chewing the tablets.

The prescribed total daily dose will be distributed across approximately 3 larger meals/snacks throughout the day to align with the approximate phosphorus intake with food. It is recognized that some subjects require different distributions in a given day due to snacks or missed meals.

If a subject eats fewer than 3 meals or snacks per day, he/she will skip the remaining doses for that day.

The proposed strategy for dose titration aims to treat to age-appropriate serum phosphorus targets; iron parameters will be monitored at each visit and repeated if elevated, and will further inform dosing decisions.

At each study visit, the dose may be adjusted on the basis of the subject's serum phosphorus level relative to the age-appropriate target. For all dosing decisions, the subject's body weight at the Screening visit (Visit 1) will be used to determine the body weight category, and the subject's age at the Screening visit (Visit 1) will be used to determine the age-appropriate serum phosphorus target.

Table 8 lists the age-appropriate serum phosphorus targets, as well as the cutoff levels for dose modification, and Table 9 provides additional details for each body weight category, as well as the maximum dose for each body weight category.

TABLE 8

| Age at Screening | Serum Phosphorus | 1.25 × ULT for Titration |
|---|---|---|
| (Visit 1) | Target (mg/dl) | Decisions (mg/dl) |
| ≥6 to ≤13 years | 3.6 to 5.8 | 7.3 |
| ≥13 to <18 years | 2.3 to 4.5 | 5.6 |

TABLE 9

| | Starting | | | | | |
|---|---|---|---|---|---|---|
| | dose: | | | | | Maxi- |
| Body | Total | Change to Ferric Citrate Daily Dose (mg) | | | | mum |
| Weight at | Daily | If serum phosphorus is | | | | Daily |
| Screening | Dose | | Within | >ULT but | ≥1.25 × | Dose |
| (kg) | (mg) | <LLT | target | <1.25 × ULT | ULT | (mg) |
| 12 to <20 | 1000 | Hold | NC | +250 | +1000 | 2500 |
| 20 to <40 | 2000 | Hold | NC | +500 | +2000 | 5000 |
| 40 to <60 | 3000 | Hold | NC | +1000 | +3000 | 9000 |
| ≥60 | 6000 | Hold | NC | +1000 | +6000 | 12000 |

LLT = lower limit of serum phosphorus target; NC = no change; ULT = upper limit of serum phosphorus target

In embodiments, a dose modification may be made based on transferrin saturation (TSAT) levels.

For example, a TSAT ≥50% may result in additional follow-up and a repeat lab assessment.

If a follow-up assessment shows that a patient has a TSAT that is <50%, then treatment with ferric citrate may continue.

If a follow-up assessment shows the patient has a TSAT ≥50%-<70%, the ferric citrate may be adjusted by ˜⅓ of the total daily dose. For example, decrease ferric citrate dose by 250 mg/day for subjects 12 to <20 kg; decrease ferric citrate dose by 500 mg/day for subjects 20 to <40 kg; decrease ferric citrate dose by 1000 mg/day for subjects 40 to <60 kg; decrease ferric citrate dose by 2000 mg/kg for subjects 260 kg.

### Example 4—Ferric Citrate in Children with Iron Deficiency Anemia Associated with Non-Dialysis Dependent Chronic Kidney Disease [KRX-0502-309]

This is a phase 3 multicenter, 24-week, randomized, 2-arm, open-label study of ferric citrate in children (ages 6 to <18 years such as ages 12 to 17 years) with IDA associated with NDD-CKD.

The study consists of a Screening period of up to 5 weeks, followed by randomization, a 24-week Treatment period, and a 30-day safety follow-up period after the last dose of study drug or standard of care treatment. After randomization, subjects assigned to standard of care (Arm 2) will remain on standard of care and subjects assigned to ferric citrate arm (Arm 1) will discontinue oral iron, if applicable and start treatment with ferric citrate. Study visits will occur every 2 weeks for the first month, and every 4 weeks thereafter.

Subjects in Arm 1 will receive ferric citrate; starting doses will be based on body weight categories. Doses may be adjusted at Weeks 4, 8, 12, 16, and 20, per dose titration guidelines to target hemoglobin (Hgb) increase, or at any time to address safety or tolerability concerns.

Subjects randomized to Arm 2 will continue to receive standard of care treatment.

Adverse events (AEs) including adverse events of special interest will be monitored throughout the treatment period. Laboratory assessments will be conducted to monitor Hgb, iron parameters, serum phosphorus levels, liver tests, and other clinically relevant laboratory values.

Ferric citrate doses may be reduced, temporarily held, or discontinued for individual subjects, if necessary, based on AEs or abnormal laboratory results including alterations in phosphorus or iron levels. For subjects in Arm 1, (ferric citrate), concomitant use of intravenous (IV) iron, or phosphate binders other than study drug will not be permitted during the study. Multivitamins containing iron are permitted. Subjects who require blood transfusions and/or subjects who begin dialysis or undergo transplantation will be withdrawn from study drug. If a subject receives a blood transfusion, the subject will immediately discontinue ferric citrate, complete the Early Termination visit assessments, and exit the study.

Main Criteria for Inclusion:
Age 6 years to <18 years (e.g., 12 to 17 years) at Screening.
Body Weight ≥12 kg (e.g., ≥40 kg) at Screening.
CKD stage 3 to 5, not on dialysis, with estimated glomerular filtration rate (eGFR)
   <60 ml/min/1.73 m² utilizing the “Bedside Schwartz” equation.
Hgb ≥8.5 and ≤11.5 g/dl at Screening.
Transferrin saturation (TSAT) ≤25% at Screening.
Ferritin ≤200 ng/ml at Screening.

Main Criteria for Exclusion:
Serum phosphorus level at Screening:
   6 to <13 years: ≤4.0 mg/dl.
   13 to <18 years: ≤2.7 mg/dl.
Liver transaminases (aspartate aminotransferase [AST] and/or alanine aminotransferase [ALT]) >3× the upper limit of normal at Screening.
Active significant GI disorder, including overt gastrointestinal (GI) bleeding or active inflammatory bowel disease.
Unable to swallow pills.
Anemia due to causes other than IDA of CKD.
IV iron therapy or blood transfusion within 4 weeks before the Screening visit.
Subjects with a functioning organ transplant.
Receipt of any investigational drug within 4 weeks before Screening.
Phosphate binder use during the Screening period.

A patient may have a documented history of CKD-related hyperphosphatemia for at least 3 months prior to the screening visit.

Ferric citrate will be supplied as:
Tablets containing 1 g ferric citrate (210 mg of ferric iron).
Tablets containing 250 mg ferric citrate (52.5 mg ferric iron).

Safety and tolerability will be assessed by monitoring:
Incidence, seriousness, and intensity of treatment-emergent AEs, including gastrointestinal adverse events of special interest.
Clinically significant laboratory abnormalities or changes in laboratory results during the Treatment period. This excludes serum Iron Indices and serum phosphorus levels (although they will be monitored).
Treatment-emergent AEs leading to discontinuation of ferric citrate.

Secondary Endpoints
Hgb change from baseline to Week 24/Early Termination visit (ET).
TSAT change from baseline to Week 24/ET.
Ferritin change from baseline to Week 24/ET.
Serum phosphorus change from baseline to Week 24/ET.
Calcium changes from baseline to Week 24/ET.
Bicarbonate changes from baseline to Week 24/ET.

Eligible subjects will be assigned a starting dose based on their body weight at Screening. Subjects will take ferric citrate orally with food (meals or snacks), or within 1 hour after eating. Tablets must be swallowed whole, without splitting, crushing, or chewing the tablets. The prescribed total daily dose will be distributed across approximately 2 or 3 meals/snacks per day.

To account for significant differences in body weight across the broad range of pediatric subjects to be enrolled, dosing will be based on body weight categories, as detailed in Tables 10 and 11. The dose is then adjusted according to Hgb level at specific study timepoints (Weeks 4, 8, 12, 16, and 20). Dosing may also be modified at any time to address AEs including abnormal laboratory results requiring dose adjustment.

TABLE 10
Summary of Ferric Citrate Dosing for Study KRX-0502-309

| Weight at | Ferric Citrate Dosing (mg/day) | | |
|---|---|---|---|
| Screening | | Titration | Maximum |
| (kg) | Starting Dose | Increments | Dose |
| 12 to <40 | 750 | 750 | 2250 |
| 40 to <60 | 1500 | 1500 | 4500 |
| ≥60 | 3000 | 3000 | 9000 |

TABLE 11
Ferric Citrate Starting Dose Assignment
for Each Body Weight Category

| Body Weight at | Starting Dose: Total | Total Daily Starting |
|---|---|---|
| Screening (kg) | Daily Dose (mg) | Dose, by Formulation |
| 12 to <40 | 750 | 250-mg Tablets: 3 tablets |
| 40 to <60 | 1500 | 250-mg Tablets: 6 tablets; |
| | | or 1-g and 250-mg (×2) |
| | | Tablets: 3 tablets |
| ≥60 | 3000 | 1-g Tablets: 3 tablets; or |
| | | 250-mg Tablets: 12 tablets |

The maximum dose of ferric citrate allowed in this study is 3 times the starting dose. The approximate weight-based equivalents of each maximum dose range from 56 to 187.5 mg/kg/day.

Subjects will take ferric citrate orally with food (meals or snacks), or within 1 hour after eating. Tablets must be swallowed whole, without splitting, crushing, or chewing the tablets.

The prescribed total daily dose will be distributed across approximately 2 or 3 meals/snacks throughout the day. Subjects will not make up a missed dose of ferric citrate if it has been more than 1 hour since their last meal/snack. If a subject occasionally eats fewer than 2 or 3 meals or snacks per day, he/she will skip the remaining doses for that day.

Ferric Citrate Dose Titration to Achieve Target Hemoglobin Increase

Dose titration may occur at Weeks 4, 8, 12, 16, and 20. If a subject's Hgb increase from baseline is ≥0.5 g/dl or Hgb level is ≥10 g/dl at the titration time point, the subject's ferric citrate dose will not be changed. If the subject's increase in Hgb from baseline is <0.5 g/dl and Hgb level is <10 g/dl at the titration time point, the subject's dose will be increased up to the maximum dose, according to the body weight category at Screening, as shown in Table 12. At each study visit, the dose may be adjusted on the basis of the subjects' Hgb level, relative to the age-appropriate target.

TABLE 12
Dose Titration Guidelines for Ferric Citrate: Dose Titration
Steps for Each Body Weight Category

| Body | Daily | | |
|---|---|---|---|
| Weight at | Dose (mg) | After 1 | Maximum Dose |
| Screening (kg) | Starting Dose | Up-Titration | After 2 Up-Titrations |
| 12 to <40 | 750 | 1500 | 2250 |
| 40 to <60 | 1500 | 3000 | 4500 |
| ≥60 | 3000 | 6000 | 9000 |

The ferric citrate dose can only be increased if the subject's serum phosphorus is above the following serum phosphorus threshold (using the subject's age at Screening):
If age is ≥6 to <13 years, serum phosphorus must be >4.0 mg/dl.
If age is ≥13 to <18 years, serum phosphorus must be >2.7 mg/dl.

These serum phosphorus thresholds are 0.4 mg/dl above the lower limit of the age-related reference ranges (established in the KDOQI Clinical Practice Guideline for Nutrition in Children with CKD [NKF 2008]) to decrease the possibility of subjects developing hypophosphatemia.

The dose of ferric citrate will be up-titrated according to the Hgb-based dose titration guideline, until the maximum dose is given or the administered dose is the maximum the subject can practically take or tolerate. Dose will also not be increased if, e.g., TSAT is high or phosphorus is low.

Modifications for Low Serum Phosphorus

Stop ferric citrate and repeat serum phosphorus assessment in 2 weeks if serum phosphorus drops below the age-appropriate serum phosphorus range established in the KDOQI Clinical Practice Guideline for Nutrition in Children with CKD [Error! Reference source not found.], specifically:

If age is ≥6 to <13 years, serum phosphorus <3.6 mg/dl.

If age is ≥13 to <18 years, serum phosphorus <2.3 mg/dl.

After 2 weeks off ferric citrate, repeat phosphorus. The ferric citrate dose can only be resumed if the subject's serum phosphorus is above the following serum phosphorus threshold (using the subject's age at Screening):

If age is ≥6 to <13 years, serum phosphorus must be >4.0 mg/dl.

If age is ≥13 to <18 years, serum phosphorus must be >2.7 mg/dl.

If the serum phosphorus is below this threshold, continue to hold ferric citrate and reassess at the next study visit. If the repeat phosphorus is above this threshold, restart ferric citrate at a dose to be determined in consultation with the Medical Monitor, and reassess at the next study visit.

Decisions about resuming dosing will take into consideration the absolute levels and trends of the serum phosphorus in the context of the prior dose changes, as well as Hgb and TSAT.

Modifications for Elevated TSAT

If TSAT is ≥70%, hold ferric citrate until repeat laboratory tests are returned. If TSAT is ≥50% and <70%, repeat laboratory tests (TSAT, ferritin, Hgb, and phosphorus) as soon as possible for confirmation while continuing ferric citrate dosing:

If the repeat TSAT is ≥70%, discontinue ferric citrate and withdraw the subject from the study.

If the repeat TSAT is ≥50 and <70%, reduce the dose of ferric citrate (specific dose to be determined in consultation with the Medical Monitor) and continue to monitor at each study visit. The dose of ferric citrate may be increased again once the TSAT is <50%.

If the repeat TSAT is <50% adjust the dose based on Hgb level.

Decisions about reducing or resuming dosing will take into consideration the absolute levels and trends of the TSAT and Hgb in the context of the prior dose changes, as well as serum phosphorus levels.

Modifications for Elevated Ferritin

An elevated ferritin level alone (i.e., ferritin ≥800 ng/ml) does not require dose interruption or dose reduction because it is a poor indicator of iron stores in patients with CKD. However, an elevated ferritin level will prompt clinical evaluation for inflammation or infection and, in review together with the Hgb and TSAT, may indicate actual iron overload and may lead to interruption or reduction of dosing.

Modifications for Another Laboratory Abnormality or Adverse Event

Another laboratory abnormality or AE may require a dose interruption or reduction. Upon resolution of the laboratory abnormality or AE, the dose will subsequently be resumed and/or increased to the highest dose tolerated prior to dose interruption or reduction.

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A pharmaceutical composition formulated as a solid oral dosage form, comprising:
an intragranular component comprising
ferric citrate present in an amount that is about 60-80 weight %;
one or more binders present in a total amount that is about 1-10 weight %;
one or more disintegrants present in a total amount that is about 1-5 weight %;
one or more fillers present in a total amount that is about 10-30 weight %; and
one or more lubricants present in a total amount that is about 0.1-2 weight %; and
an extragranular component comprising
one or more glidants present in a total amount that is about 0.1-2 weight %; and
one or more lubricants present in a total amount that is about 0.1-2 weight %;
wherein the weight % is determined based on the total weight of the tablet.

2. The pharmaceutical composition of claim 1, wherein the one or more binders of the intragranular component are present in a total amount that is about 3-10, 3-9, 3-8, 3-6, 3-5, 4-10, 4-9, 4-8, 4-7, or 4-6 weight %.

3. The pharmaceutical composition of claim 1 or 2, wherein the one or more binders of the intragranular component are selected from the group consisting of hydroxypropyl cellulose (HPC), hydroxypropylmethyl cellulose (HPMC), sodium alginate, alginic acid, guar gum, acacia gum, xanthan gum, carbolpol, cellulose gum (carboxy methyl cellulose), ethyl cellulose, maltodextrin, PVP/VA, povidone, microcrystalline cellulose, starch (partially or fully pregelatinized starch), methyl cellulose, and copovidone.

4. The pharmaceutical composition of claim 3, wherein the intragranular component comprises a binder that is copovidone.

5. The pharmaceutical composition of any one of claims 1-4, wherein the one or more disintegrants of the intragranular component are present in a total amount that is about 1-2, 2-3, 3-4, or 4-5 weight %.

6. The pharmaceutical composition of any one of claims 1-5, wherein the one or more disintegrants of the intragranular component are selected from the group consisting of croscarmellose sodium, crospovidone, sodium starch glycolate, starch, and microcrystalline cellulose.

7. The pharmaceutical composition of any one of claims 1-6, wherein the one or more fillers of the intragranular component are present in a total amount that is about 10-25, 10-20, 15-25, 15-30, 20-30, or 20-25 weight %.

8. The pharmaceutical composition of any one of claims 1-7, wherein the one or more fillers of the intragranular component are selected from microcrystalline cellulose, starches, partially pregelatinized starches, sorbitol powder, mannitol powder, lactose, hydroxypropyl methyl cellulose, hydroxypropyl cellulose, maltodextrins, dried glucose syrup, and dextrose mono & anhydrous.

9. The pharmaceutical composition of any one of claims 1-8, wherein
the one or more lubricants of the intragranular component are present in a total amount of about 0.1-1 weight %; and/or
the one or more lubricants of the extragranular component are present in a total amount of about 0.1-1 weight %.

10. The pharmaceutical composition of any one of claims 1-9, wherein the one or more lubricants of the intragranular and/or extragranular components are selected from the group consisting of magnesium stearate, calcium stearate, sodium stearyl fumarate, polyethylene glycol, sodium lauryl sulfate, talc, mineral oil, leucine, and poloxamer.

11. The pharmaceutical composition of claim 10, wherein the intragranular and extragranular components comprise a lubricant that is magnesium stearate.

12. The pharmaceutical composition of claim 10, wherein the intragranular and extragranular components comprise a lubricant that is calcium stearate.

13. The pharmaceutical composition of any one of claims 1-12, wherein the one or more glidants of the extragranular component are present in a total amount of about 0.1-1 weight %.

14. The pharmaceutical composition of any one of claims 1-13, wherein the one or more glidants of the extragranular component are selected from the group consisting of hydrophilic fumed silica, colloidal silicon dioxide, starch, talc, and magnesium stearate.

15. The pharmaceutical composition of claim 14, wherein the extragranular component comprises a glidant that is hydrophilic fumed silica or colloidal silicon dioxide.

16. A pharmaceutical composition comprising:
an intragranular component comprising
ferric citrate present in an amount that is about 60-80 weight %;
two or more excipients selected from the group consisting of copovidone, microcrystalline cellulose, and crospovidone, wherein said excipients are present in a total amount that is about 20-35 weight %; and
magnesium stearate or calcium stearate present in an amount that is about 0.1-2 weight %; and
an extragranular component comprising
hydrophilic fumed silica or colloidal silicon dioxide present in an amount that is about 0.1-2 weight %; and
magnesium stearate or calcium stearate present in an amount that is about 0.1-2 weight %;
wherein the weight % is determined based on the total weight of the tablet.

17. The pharmaceutical composition of any one of claims 1-16, wherein the intragranular component comprises copovidone, microcrystalline cellulose, and crospovidone.

18. The pharmaceutical composition of any one of claims 1-17, wherein ferric citrate is present in an amount that is about 60-75, 65-80, 65-75, 70-80, or 70-75 weight %.

19. The pharmaceutical composition of any one of claims 1-18, wherein ferric citrate is present in an amount that is about 65-75 or 70-75 weight %.

20. The pharmaceutical composition of any one of claims 1-19, comprising about 100-1000 mg ferric citrate.

21. The pharmaceutical composition of claim 20, comprising about 100-900, 100-800, 100-700, 100-600, 100-500, 100-400, 100-300, 100-200, 200-900, 200-800, 200-700, 200-600, 200-500, 200-400, 200-300, 300-900, 400-800, 400-700, 400-600, 400-500, 500-900, 500-800, 500-700, or 500-600 mg ferric citrate.

22. The pharmaceutical composition of claim 20 or 21, comprising about 100-500, 200-500, or 300-500 mg ferric citrate or about 50, 100, 150, 200, 250, 300, 350, 400, 450, or 500 mg ferric citrate.

23. The pharmaceutical composition of any one of claims 20-22, comprising about 250 mg ferric citrate.

24. The pharmaceutical composition of any one of claims 1-23, formulated as a tablet.

25. The pharmaceutical composition of claim 24, wherein the tablet further comprises a coating.

26. The pharmaceutical composition of claim 25, wherein said coating comprises hydroxypropyl methylcellulose (HPMC) as the binder.

27. The pharmaceutical composition of claim 25 or 26, wherein said coating is Opadry® Purple.

28. The pharmaceutical composition of any one of claims 25-27, wherein the coating does not comprise polyvinyl alcohol (PVA) or polyvinylpyrrolidone (PVP) as a binder.

29. The pharmaceutical composition of any one of claims 24-28, wherein the tablet comprises:
an intragranular component comprising
ferric citrate in an amount that is about 65-75 weight %;
a binder in an amount that is about 3-8 weight %;
a filler in an amount that is about 15-25 weight %;
a disintegrant in an amount that is about 1-3 weight %; and
a lubricant in an amount that is about 0.1-0.5 weight %; and
an extragranular component comprising
one or more glidants in a total amount that is about 0.1-0.5 weight %; and
one or more lubricants in a total amount that is about 0.3-0.8 weight %; and
an optional coating in an amount that is about 1-5 weight %, wherein said coating comprises a non-polyvinyl alcohol binder; and
wherein the weight % is determined based on the total weight of the tablet.

30. The pharmaceutical composition of any one of claims 24-29, wherein the tablet comprises:
an intragranular component comprising
ferric citrate in an amount that is about 65-75 weight %;
copovidone in an amount that is about 3-8 weight %;
microcrystalline cellulose in an amount that is about 15-25 weight %;
crospovidone in an amount that is about 1-3 weight %; and
magnesium stearate in an amount that is about 0.1-0.5 weight %; and
an extragranular component comprising
colloidal silicon dioxide in a total amount that is about 0.1-0.5 weight %; and
magnesium stearate in a total amount that is about 0.3-0.8 weight %; and
an optional coating in an amount that is about 1-5 weight %, wherein said coating comprises a non-polyvinyl alcohol binder; and
wherein the weight % is determined based on the total weight of the tablet.

31. The pharmaceutical composition of any one of claims 24-30, wherein the tablet comprises:
an intragranular component comprising
about 250 mg (±10% or ±5%) ferric citrate;
about 17.9 mg (±10% or ±5%) copovidone;
about 71.6 mg (±10% or ±5%) microcrystalline cellulose;
about 7.1 mg (±10% or ±5%) crospovidone; and
about 0.9 mg (±10% or ±5%) magnesium stearate; and
an extragranular component comprising
about 0.7 mg (±10% or ±5%) colloidal silicon dioxide; and
about 1.8 mg (±10% or ±5%) magnesium stearate; and
about 14.0 g (±10% or ±5%) of a coating, wherein said coating comprises a non-polyvinyl alcohol binder.

32. The pharmaceutical composition of any one of claims 24-31, wherein a tablet coating comprises hydroxypropyl methylcellulose (HPMC) as the binder.

33. The pharmaceutical composition of any one of claims 24-32, wherein the tablet coating is Opadry® Purple.

34. The pharmaceutical composition of any one of claims 24-33, wherein the tablet is formulated for immediate release of the ferric citrate.

35. The pharmaceutical composition of any one of claims 24-34, wherein the tablet has a total weight of about 200-500 mg, 250-450 mg, or 300-400 mg.

36. The pharmaceutical composition of any one of claims 24-35, wherein the tablet has a hardness of about 10-20 or 12-18 kp.

37. The pharmaceutical composition of any one of claims 24-36, wherein the tablet has a friability that is ≤ about 1%.

38. The pharmaceutical composition of any one of claims 24-37, wherein the tablet has a disintegration time of ≤ about 20 or 15 minutes.

39. The pharmaceutical composition of any one of claims 24-38, wherein the tablet has a BET specific surface area greater than 5 m²/g.

40. The pharmaceutical composition of any one of claims 24-38, wherein the tablet has a BET specific surface area greater than 10 m²/g.

41. The pharmaceutical composition of any one of claims 24-38, wherein the tablet has a BET specific surface area greater than 20 m²/g.

42. The pharmaceutical composition of claim 41, wherein the BET specific surface area ranges from 20 m²/g to 40 m²/g, 25 m²/g to 35 m²/g, or 25 m²/g to 30 m²/g.

43. The pharmaceutical composition of any one of claims 1-23, formulated for administration as granules or a powder.

44. A method for the prophylaxis or treatment of hyperphosphatemia in a subject in need thereof, comprising administering to the subject an effective amount of ferric citrate, wherein the subject is ≤ about 18 years of age, and wherein the subject has chronic kidney disease.

45. The method of claim 44, wherein the subject is about 6 to <18 years of age.

46. The method of claim 44 or 45, wherein the subject receives a weight-based dose of ferric citrate.

47. The method of claim 46, wherein
a subject of about 12 to <20 kg receives an initial daily dose of ferric citrate of about 1000 mg;
a subject of about 20 to <40 kg receives an initial daily dose of ferric citrate of about 2000 mg;
a subject of about 40 to <60 kg receives an initial daily dose of ferric citrate of about 3000 mg; or
a subject of about ≥60 kg receives an initial daily dose of ferric citrate of about 6000 mg.

48. The method of claim 46 or 47, wherein
a subject of about 12 to <20 kg receives a maximum daily dose of ferric citrate of about 1000 mg, wherein the daily dose is optionally adjusted by increments of about 250 mg or about 1000 mg;
a subject of about 20 to <40 kg receives a maximum daily dose of ferric citrate of about 5000 mg, wherein the daily dose is optionally adjusted by increments of about 500 mg or about 2000 mg;
a subject of about 40 to <60 kg receives a maximum daily dose of ferric citrate of about 9000 mg, wherein the daily dose is optionally adjusted by increments of about 1000 mg or about 3000 mg; or
a subject of about ≥60 kg receives a maximum daily dose of ferric citrate of about 12000 mg, wherein the daily dose is optionally adjusted by increments of about 1000 mg or about 6000 mg.

49. The method of any one of claims 44-48, wherein the subject is on dialysis.

50. A method of treating iron deficiency anemia in a subject in need thereof, comprising administering to the subject an effective amount of ferric citrate, wherein the subject is ≤ about 18 years of age, and wherein the subject has chronic kidney disease.

51. The method of claim 50, wherein the subject is about 6 to <18 years of age or about 12 to 17 years of age.

52. The method of claim 50 or 51, wherein the subject is not on dialysis.

53. The method of any one of claims 50-52, wherein the subject receives a weight-based dose of ferric citrate.

54. The method of claim 53, wherein
a subject of about 12 to <40 kg receives an initial daily dose of about 750 mg ferric citrate;
a subject of about 40 to <60 kg receives an initial daily dose of about 1500 mg ferric citrate; or
a subject of about ≥60 kg receives an initial daily dose of about 3000 mg ferric citrate.

55. The method of claim 53 or 54, wherein
a subject of about 12 to <40 kg receives a maximum daily dose of about 2250 mg ferric citrate, wherein the daily dose is optionally adjusted by increments of about 750 mg;
a subject of about 40 to <60 kg receives a maximum daily dose of about 4500 mg ferric citrate, wherein the daily dose is optionally adjusted by increments of about 1500 mg; or
a subject of about ≥60 kg receives a maximum daily dose of about 9000 mg ferric citrate, wherein the daily dose is optionally adjusted by increments of about 3000 mg.

56. The method of any one of claims 50-55, wherein the subject is about 12 to 17 years of age and/or about ≥40 kg.

57. The method of any one of claims 44-56, comprising administration of the ferric citrate as the pharmaceutical composition of any one of claims 1-43.

58. The method of claim 57, wherein the subject is of about 6-18 years of age or about 12 to 17 years of age.

59. The method of claim 58, wherein the pharmaceutical composition is administered as a tablet.

60. The method of claim 59, comprising administration of the pharmaceutical composition of any one of claims 24-42.

61. A method of preparing the pharmaceutical composition of any one of claims 1-43, comprising a first step of blending the ferric citrate, the one or more binders, the one or more fillers, and the one or more disintegrants, of the intragranular phase to form a first pre-blend, and wherein the components are optionally screened prior to blending.

62. The method of claim 61, further comprising blending the one or more lubricants of the intragranular phase with the first pre-blend to form the second pre-blend, wherein the one or more lubricants are optionally screened prior to blending.

63. The method of claim 61 or 62, wherein the blended material is granulated by dry granulation process to form granules of suitable particle size distribution.

64. The method of claim 61, comprising a second step of blending the granules with the one or more glidants and the one or more lubricants of the extragranular component to form the blend, optionally wherein the one or more glidants and the one or more lubricants are screened prior to blending.

65. The method of claim 64, wherein the blend is compressed to form a tablet.

66. The method of claim 65, wherein the compressed tablets are coated with suitable coating material consisting of cellulosic product.
